(19) United States
(12) Patent Application Publication
(10) Pub. No.: US 2006/0205838 A1
(43) Pub. Date: Sep. 14, 2006

(54) Hardenable antimicrobial dental compositions and methods

(76) Inventors: Bhaskar V. Velamakanni, Woodbury, MN (US); Sumita B. Mitra, West St. Paul, MN (US); Danli Wang, Shoreview, MN (US); Matthew T. Scholz, Woodbury, MN (US); Steven M. Aasen, Woodbury, MN (US)

Correspondence Address: 3M INNOVATIVE PROPERTIES COMPANY, PO BOX 33427, ST. PAUL, MN, 55133-3427, US

(21) Appl. No.: 11/077,865

(22) Filed: Mar. 10, 2005

(51) Int. Cl. C09K 3/00; A61K 6/083

(52) U.S. Cl. 523/115; 106/35

(57) ABSTRACT

The present application provides dental compositions, methods of making, and methods of using dental compositions that include an antimicrobial lipid component and a hardenable component.

### BACKGROUND

Due to an alarming increase in drug-resistant bacterial infections, antibiotic use in oral care has been limited for the management of active infectious diseases. Typically, antiseptics and disinfectants have been used in the oral environment in the war against disease-causing microorganisms. For example, glutaraldehyde, chlorhexidine, quaternary ammonium salts, triclosan, etc., are often used for oral hygiene in oral rinses, dentrifices, and dental restorative materials such as etchants, varnishes, adhesives, etc. Recently, reactive polymers of quaternary ammonium salts are being used as immobilized antimicrobial dental adhesives.

Such antimicrobial materials often have limited effectiveness against a narrow spectrum of pathogenic bacteria. For example, cationic quaternary ammonium salts tend to chelate with metal ions in the oral cavity and lose their effectiveness. Thus, new dental compositions having antimicrobial activity are needed.

### SUMMARY OF THE INVENTION

The present invention provides dental compositions having antimicrobial activity that are useful for local/topical treatment (therapeutic or prophylactic) of conditions that are caused, or aggravated by, microorganisms. More specifically, dental compositions of the present invention are useful for preparing dental materials and articles that are effective against one or more microbes (including viruses, bacteria, yeast, mold, fungi, micoplasma, and protozoa), particularly in the oral environment.

In one embodiment, the present invention provides a dental composition that includes: an effective amount of an antimicrobial lipid component including a (C7-C12)saturated fatty acid ester of a polyhydric alcohol, a (C8-C22)unsaturated fatty acid ester of a polyhydric alcohol, a (C7-C12)saturated fatty ether of a polyhydric alcohol, a (C8-C22)unsaturated fatty ether of a polyhydric alcohol, an alkoxylated derivative thereof, or combinations thereof, wherein the alkoxylated derivative has less than 5 moles of alkoxide per mole of polyhydric alcohol; with the proviso that for polyhydric alcohols other than sucrose, the esters comprise monoesters and the ethers comprise monoethers, and for sucrose the esters comprise monoesters, diesters, or combinations thereof, and the ethers comprise monoethers, diethers, or combinations thereof; and a hardenable component.

For certain embodiments, the hardenable component includes acid functionality. For certain embodiments, the acid functionality includes carboxylic acid functionality, phosphoric acid functionality, phosphonic acid functionality, sulfonic acid functionality, or combinations thereof.

For certain embodiments, compositions of the present invention also include an initiator system.

For certain embodiments, the hardenable component includes an ethylenically unsaturated compound. For certain embodiments, the ethylenically unsaturated compound is selected from the group consisting of an ethylenically unsaturated compound with acid functionality, an ethylenically unsaturated compound without acid functionality, and combinations thereof. For certain embodiments, the ethylenically unsaturated compound is a (meth)acrylate compound.

For certain embodiments, the hardenable component includes a glass ionomer cement. For certain embodiments, the glass ionomer cement is a resin-modified glass ionomer cement.

For certain embodiments, the hardenable component includes a polyether, a polysiloxane, or combinations thereof.

For certain embodiments, the hardenable component includes an epoxide, a vinyl ether, or combinations thereof.

For certain embodiments, the antimicrobial lipid component includes glycerol monolaurate, glycerol monocaprate, glycerol monocaprylate, propylene glycol monolaurate, propylene glycol monocaprate, propylene glycol monocaprylate, or combinations thereof.

For certain embodiments, the antimicrobial lipid component is present in an amount of at least 0.1 wt-%.

For certain embodiments, the antimicrobial lipid component includes a monoester of a polyhydric alcohol, a monoether of a polyhydric alcohol, or an alkoxylated derivative thereof, and the antimicrobial lipid component further includes no greater than 15 wt-%, based on the total weight of the antimicrobial lipid component, of a di- or tri-ester, a di- or tri-ether, alkoxylated derivative thereof, or combinations thereof.

For certain embodiments, dental compositions of the present invention can further include an effective amount of an enhancer component distinct from the antimicrobial lipid component. For certain embodiments, the enhancer component can include a carboxylic acid. For certain embodiments, the enhancer component can include an alpha-hydroxy acid. For certain embodiments, the enhancer component includes an alpha-hydroxy acid, a beta-hydroxy acid, a chelating agent, a (C1-C4)alkyl carboxylic acid, a (C6-C12)aryl carboxylic acid, a (C6-C12)aralkyl carboxylic acid, a (C6-C12)alkaryl carboxylic acid, a phenolic compound, a (C1-C10)alkyl alcohol, an ether glycol, or combinations thereof. For certain embodiments, the total concentration of the enhancer component relative to the total concentration of lipid component is within a range of 10:1 to 1:300, on a weight basis.

For certain embodiments, dental compositions of the present invention can further include an effective amount of a surfactant component distinct from the antimicrobial lipid component. For certain embodiments, the surfactant component can include a sulfonate surfactant, a sulfate surfactant, a phosphonate surfactant, a phosphate surfactant, a poloxamer surfactant, a cationic surfactant, or mixtures thereof. For certain embodiments, the surfactant component can include a sulfonate surfactant, a sulfate surfactant, a poloxamer surfactant, or mixtures thereof. For certain embodiments, the surfactant component is dioctyl sodium sulfosuccinate. For certain embodiments, the surfactant component is a poloxamer including a copolymer of polyethylene oxide and polypropylene oxide. For certain embodiments, the total concentration of the surfactant component to the total concentration of antimicrobial lipid component is within a range of 5:1 to 1:100, on a weight basis.

For certain embodiments, dental compositions of the present invention can further include a filler.

For certain embodiments, dental compositions of the present invention are selected from the group consisting of dental adhesives, orthodontic adhesives, composites, restoratives, dental cements, orthodontic cements, sealants, coatings, impression materials, filling materials, and combinations thereof.

The present invention also provides a method of preparing a dental article. The method includes: combining an antimicrobial lipid component and a hardenable component to form a dental composition of the present invention; and hardening the composition to fabricate a dental article selected from the group consisting of crowns, bridges, veneers, inlays, onlays, fillings, mill blanks, impression materials, orthodontic devices, prostheses, and finishing or polishing devices.

Definitions

As used herein, a “hardenable” component refers to one that is capable of polymerization and/or crosslinking reactions including, for example, photopolymerization reactions and chemical polymerization techniques (e.g., ionic reactions or chemical reactions forming radicals effective to polymerize ethylenically unsaturated compounds, oxirane compounds, etc.) involving one or more compounds capable of hardening. Hardening reactions also include acid-base setting reactions such as those common for cement forming compositions (e.g., zinc polycarboxylate cements, glass-ionomer cements, etc.).

As used herein, “dental composition” refers to hardenable compositions used in the oral environment including, for example, dental adhesives, orthodontic adhesives, composites, restoratives, dental cements, orthodontic cements, sealants, coatings, impression materials, filling materials, and combinations thereof. In some embodiments, dental compositions of the present invention including a hardenable component can be hardened to fabricate a dental article selected from the group consisting of crowns, bridges. veneers, inlays, onlays, fillings, mill blanks, impression materials, orthodontic devices, prostheses (e.g., partial or full dentures), and finishing or polishing devices as used for dental prophylaxis or restorative treatments (e.g., prophy agents such as cups, brushes, polishing agents). As used herein, a “dental adhesive” refers to a non-filled or a lightly filled dental composition (e.g., less than 40% by weight filler), which is typically used to adhere a curable dental material (e.g., a filling material) to a tooth surface. After hardening, the dental compositions are typically not tacky or sticky and therefore would not be in the class of materials known as pressure sensitive adhesives (PSAs).

As used herein, a “dental cement” refers to a highly filled dental composition (e.g., at least 40% by weight filler), which is typically used to adhere a pre-formed or pre-cured dental article (e.g., an inlay, an onlay, a crown, or the like) to a tooth surface.

As used herein, an “orthodontic cement” refers to a composition that is typically used as a pre-treatment on a dental structure (e.g., a tooth) to adhere an orthodontic appliance (e.g., a band) to the dental structure.

As used herein, an “orthodontic adhesive” refers to a highly filled composition (e.g., at least 40% by weight filler), which is typically used to adhere an orthodontic appliance (e.g., a bracket) to a dental structure (e.g., tooth) surface. Generally, the dental structure surface is pre-treated, e.g., by etching, priming, and/or applying an adhesive to enhance the adhesion of the orthodontic adhesive or orthodontic cement to the dental structure surface.

As used herein, “impression material” refers to a material that is used in a softened or low viscosity form (uncured state) to make an accurate impression of hard and/or soft tissues within the oral cavity, and then cured to a hard or high viscosity form (cured state) that represents a negative model of the hard and/or soft tissues. In the cured state, the impression material needs to be able to receive a low viscosity material (e.g., a gypsum slurry), which after setting (i.e., hardening) represents a positive model of the hard and/or soft tissues of the mouth.

As used herein, a “filling material” refers to a composition that is used to fill a defect in the tooth to restore its functionality. Often such filling materials are two part systems that cure gradually when these parts are mixed. Such materials could be glass ionomers, resin modified glass ionomers or self-curing resin-based composites typically with methacrylates or epoxy matrices.

As used herein, “(meth)acryl” is a shorthand term referring to “acryl” and/or “methacryl.” For example, a “(meth)acryloxy” group is a shorthand term referring to either an acryloxy group (i.e., CH₂═CHC(O)O—) and/or a methacryloxy group (i.e., CH₂═C(CH₃)C(O)O—).

“Effective amount” means the amount of the antimicrobial lipid component plus the enhancer component (when present in a composition) and/or the surfactant component (when present in a composition), as a whole, provides an antimicrobial (including, for example, antiviral, antibacterial, or antifungal) activity that reduces, prevents, or eliminates one or more species of microbes such that an acceptable level of the microbe results. Typically, this is a level low enough not to cause clinical symptoms, and is desirably a non-detectable level. It should be understood that in the compositions of the present invention, the concentrations or amounts of the components, when considered separately, may not kill to an acceptable level, or may not kill as broad a spectrum of undesired microorganisms, or may not kill as fast; however, when used together such components provide an enhanced (preferably synergistic) antimicrobial activity (as compared to the same components used alone under the same conditions).

“Enhancer” means a component that enhances the effectiveness of the antimicrobial lipid component such that when the composition less the antimicrobial lipid component and the composition less the enhancer component are used separately, they do not provide the same level of antimicrobial activity as the composition as a whole. For example, an enhancer component in the absence of the antimicrobial lipid component may not provide any appreciable antimicrobial activity. The enhancing effect can be with respect to the level of kill, the speed of kill, and/or the spectrum of microorganisms killed, and may not be seen for all microorganisms. In fact, an enhanced level of kill is most often seen in Gram negative bacteria such as Escherichia coli. An enhancer may be a synergist such that when combined with the remainder of the composition, the composition as a whole displays an activity that is greater than the sum of the activity of the composition less the enhancer component and the composition less the antimicrobial lipid component.

“Microorganism” or “microbe” or “microorganism” refers to bacteria, yeast, mold, fungi, protozoa, mycoplasma, as well as viruses (including lipid enveloped RNA and DNA viruses).

“Antiseptic” means a chemical agent that kills pathogenic and non-pathogenic microorganisms. Preferred antiseptics exhibit at least a 4 log reduction of both P. aeruginosa and S. aureus in 60 minutes from an initial inoculum of 1-3×10⁷ cfu/ml when tested in Mueller Hinton broth at 35° C. at a concentration of 0.25 wt-% in a Rate of Kill assay using an appropriate neutralizer as described in “The Antimicrobial Activity in vitro of chlorhexidine, a mixture of isothiazolinones (Kathon CG) and cetyl trimethyl ammonium bromide (CTAB),” G. Nicoletti et al., Journal of Hospital Infection, 23, 87-111 (1993). Antiseptics generally interfere with the cellular metabolism and/or the cell envelope. Antiseptics are sometimes referred to as disinfectants, especially when used to treat hard surfaces.

“Antimicrobial lipid” means an antiseptic having at least one (C6)alkyl or alkylene chain (preferably at least one (C7) chain and more preferably at least one (C8) chain), and preferably having a solubility in water of no greater than 1.0 gram per 100 grams (1.0 g/100 g) deionized water. Preferred antimicrobial lipids have a solubility in water of no greater than 0.5 g/100 g deionized water, more preferably, no greater than 0.25 g/100 g deionized water, and even more preferably, no greater than 0.10 g/100 g deionized water. Solubilities are determined using radiolabeled compounds as described under “Conventional Solubility Estimations” in Solubility of Long-Chain Fatty Acids in Phosphate Buffer at pH 7.4, Henrik Vorum et al., in Biochimica et. Biophysica Acta., 1126, 135-142 (1992). Preferred antimicrobial lipids have a solubility in deionized water of at least 100 micrograms (μg) per 100 grams deionized water, more preferably, at least 500 μg/100 g deionized water, and even more preferably, at least 1000 μg/100 g deionized water. The antimicrobial lipids preferably have a hydrophile/lipophile balance (HLB) of at most 6.2, more preferably at most 5.8, and even more preferably at most 5.5. The antimicrobial lipids preferably have an HLB of at least 3, preferably at least 3.2, and even more preferably at least 3.4.

“Fatty” as used herein refers to a straight or branched chain alkyl or alkylene moiety having at least 6 (odd or even number) carbon atoms, unless otherwise specified.

The term “comprises” and variations thereof do not have a limiting meaning where these terms appear in the description and claims.

As used herein, “a,” “an,” “the,” “at least one,” and “one or more” are used interchangeably. The term “and/or” means one or all of the listed elements.

Also herein, the recitations of numerical ranges by endpoints include all numbers subsumed within that range (e.g., 1 to 5 includes 1, 1.5, 2, 2.75, 3, 3.80, 4, 5, etc.).

The above summary of the present invention is not intended to describe each disclosed embodiment or every implementation of the present invention. The description that follows more particularly exemplifies illustrative embodiments. In several places throughout the application, guidance is provided through lists of examples, which examples can be used in various combinations. In each instance, the recited list serves only as a representative group and should not be interpreted as an exclusive list.

### DETAILED DESCRIPTION OF ILLUSTRATIVE EMBODIMENTS

The present invention provides dental compositions that include an antimicrobial lipid component. Methods of making and using such dental compositions are also provided. Such compositions have antimicrobial activity and are useful for local/topical treatment (therapeutic or prophylactic) of conditions that are caused, or aggravated by, microorganisms. More specifically, such compositions are useful for preparing dental materials and articles that are effective against one or more microbes (including viruses, bacteria, yeast, mold, fungi, micoplasma, and protozoa), particularly in the oral environment.

The present invention provides dental compositions that include an antimicrobial lipid component and a hardenable component. Such dental compositions are typically prepared by combining the antimicrobial lipid component with the hardenable component. Other optional components of the dental compositions of the present invention include enhancers, surfactants, and fillers, for example.

The dental compositions of the present invention (both before and after hardening) can have antimicrobial activity and preferably are active against a broad spectrum of bacteria including Gram-positive and Gram-negative bacteria. Certain preferred embodiments have good to excellent activity against Streptococcus mutans (S. mutans) bacteria. S. mutans has the tendency to adhere to hard surfaces, such as teeth, forming a biofilm or plaque. Such colonization can eventually lead to a number of undesirable clinical side effects that include origination of caries, calcified plaque, irritation of gum tissue leading up to periodontal diseases, etc. Therefore, some of the clinical benefits of using antimicrobial agents in dental materials, such as adhesives or composites, are not only to kill harmful bacteria in the oral cavity but also to suppress the formation of biofilm and secondary caries under a restoration.

As detailed in the Examples Section, effective amounts of certain present invention compositions (before curing) have provided greater than 2 log reductions, preferably greater than 3 log reductions, and more preferably greater than 4 log reductions of S. mutans as evaluated by the Bacteria Kill Rate Test Method described herein. Effective amounts of certain present invention compositions (after curing) have provided greater than 3 log reductions, preferably greater than 5 log reductions, and more preferably greater than 8 log reductions of S. mutans as evaluated by the Extended Disinfectant Test Method described herein. Additionally, hardened discs prepared from certain embodiments showed a propensity to resist adherence of biofilm/plaque (attributed to S. mutans) to the surface of the discs as evaluated by the S. Mutans Bacteria Adherence Test Method described herein.

Antimicrobial Lipid Component

The antimicrobial lipid component is that component of the composition that provides at least part of the antimicrobial activity. That is, the antimicrobial lipid component has at least some antimicrobial activity for at least one microorganism. It is generally considered the main active component of the compositions of the present invention.

In certain embodiments, the antimicrobial lipid preferably has a solubility in water of no greater than 1.0 gram per 100 grams (1.0 g/100 g) deionized water. More preferred antimicrobial lipids have a solubility in water of no greater than 0.5 g/100 g deionized water, even more preferably, no greater than 0.25 g/100 g deionized water, and even more preferably, no greater than 0.10 g/100 g deionized water. Preferred antimicrobial lipids have a solubility in deionized water of at least 100 micrograms (μg) per 100 grams deionized water, more preferably, at least 500 μg/100 g deionized water, and even more preferably, at least 1000 μg/100 g deionized water.

The antimicrobial lipids preferably have a hydrophile/lipophile balance (HLB) of at most 6.2, more preferably at most 5.8, and even more preferably at most 5.5. The antimicrobial lipids preferably have an HLB of at least 3, preferably at least 3.2, and even more preferably at least 3.4.

Preferred antimicrobial lipids are uncharged and have an alkyl or alkenyl hydrocarbon chain containing at least 7 carbon atoms.

In certain embodiments, the antimicrobial lipid component preferably includes one or more fatty acid esters of a polyhydric alcohol, fatty ethers of a polyhydric alcohol, or alkoxylated derivatives thereof (of either or both of the ester and ether), or combinations thereof. More specifically and preferably, the antimicrobial component is selected from the group consisting of a (C7-C14)saturated fatty acid ester of a polyhydric alcohol (preferably, a (C7-C12)saturated fatty acid ester of a polyhydric alcohol, and more preferably, a (C8-C12)saturated fatty acid ester of a polyhydric alcohol), a (C8-C22)unsaturated fatty acid ester of a polyhydric alcohol (preferably, a (C12-C22)unsaturated fatty acid ester of a polyhydric alcohol), a (C7-C14)saturated fatty ether of a polyhydric alcohol (preferably, a (C7-C12)saturated fatty ether of a polyhydric alcohol, and more preferably, a (C8-C12)saturated fatty ether of a polyhydric alcohol), a (C8-C22)unsaturated fatty ether of a polyhydric alcohol (preferably, a (C12-C22)unsaturated fatty ether of a polyhydric alcohol), an alkoxylated derivative thereof, and combinations thereof. Preferably, the esters and ethers are monoesters and monoethers, unless they are esters and ethers of sucrose in which case they can be monoesters, diesters, monoethers, or monoethers. Various combinations of monoesters, diesters, monoethers, and diethers can be used in a composition of the present invention.

A fatty acid ester of a polyhydric alcohol is preferably of the formula (R¹—C(O)—O)ₙ—R², wherein R¹ is the residue of a (C7-C14)saturated fatty acid (preferably, a (C7-C12)saturated fatty acid, and more preferably, a (C8-C12)saturated fatty acid), or a (C8-C22)unsaturated fatty acid (preferably, a (C12-C22)unsaturated, including polyunsaturated, fatty acid), R² is the residue of a polyhydric alcohol (typically and preferably, glycerin, propylene glycol, and sucrose, although a wide variety of others can be used including pentaerythritol, sorbitol, mannitol, xylitol, etc.), and n=1 or 2. The R² group includes at least one free hydroxyl group (preferably, residues of glycerin, propylene glycol, or sucrose). Preferred fatty acid esters of polyhydric alcohols are esters derived from C7, C8, C9, C10, C11, and C12 saturated fatty acids. For embodiments in which the polyhydric alcohol is glycerin or propylene glycol, n=1, although when it is sucrose, n=1 or 2.

Exemplary fatty acid monoesters include, but are not limited to, glycerol monoesters of lauric (monolaurin), caprylic (monocaprylin), and capric (monocaprin) acid, and propylene glycol monoesters of lauric, caprylic, and capric acid, as well as lauric, caprylic, and capric acid monoesters of sucrose. Other fatty acid monoesters include glycerin and propylene glycol monoesters of oleic (18:1), linoleic (18:2), linolenic (18:3), and arachonic (20:4) unsaturated (including polyunsaturated) fatty acids. As is generally known, 18:1, for example, means the compound has 18 carbon atoms and 1 carbon-carbon double bond. Preferred unsaturated chains have at least one unsaturated group in the cis isomer form. In certain preferred embodiments, the fatty acid monoesters that are suitable for use in the present composition include known monoesters of lauric, caprylic, and capric acid, such as that known as GML or the trade designation LAURICIDIN (the glycerol monoester of lauric acid commonly referred to as monolaurin or glycerol monolaurate), glycerol monocaprate, glycerol monocaprylate, propylene glycol monolaurate, propylene glycol monocaprate, propylene glycol monocaprylate, and combinations thereof.

Exemplary fatty acid diesters of sucrose include, but are not limited to, lauric, caprylic, and capric diesters of sucrose as well as combinations thereof.

A fatty ether of a polyhydric alcohol is preferably of the formula (R³—O)ₙ—R⁴, wherein R³ is a (C7-C14)saturated aliphatic group (preferably, a (C7-C12)saturated aliphatic group, and more preferably, a (C8-C12)saturated aliphatic group), or a (C8-C22)unsaturated aliphatic group (preferably, a (C12-C22)unsaturated, including polyunsaturated, aliphatic group), R⁴ is the residue of glycerin, sucrose, or propylene glycol, and n=1 or 2. For glycerin and propylene glycol n=1, and for sucrose n=1 or 2. Preferred fatty ethers are monoethers of (C7-C14)alkyl groups (more preferably, (C7-C12)alkyl groups, and even more preferably, (C8-C12)alkyl groups).

Exemplary fatty monoethers include, but are not limited to, laurylglyceryl ether, caprylglycerylether, caprylylglyceryl ether, laurylpropylene glycol ether, caprylpropyleneglycol ether, and caprylylpropyleneglycol ether. Other fatty monoethers include glycerin and propylene glycol monoethers of oleyl (18:1), linoleyl (18:2), linolenyl (18:3), and arachonyl (20:4) unsaturated and polyunsaturated fatty alcohols. In certain preferred embodiments, the fatty monoethers that are suitable for use in the present composition include laurylglyceryl ether, caprylglycerylether, caprylyl glyceryl ether, laurylpropylene glycol ether, caprylpropyleneglycol ether, caprylylpropyleneglycol ether, and combinations thereof. Unsaturated chains preferably have at least one unsaturated bond in the cis isomer form.

The alkoxylated derivatives of the aforementioned fatty acid esters and fatty ethers (e.g., one which is ethoxylated and/or propoxylated on the remaining alcohol group(s)) also have antimicrobial activity as long as the total alkoxylate is kept relatively low. Preferred alkoxylation levels are disclosed in U.S. Pat. No. 5,208,257 (Kabara). In the case where the esters and ethers are ethoxylated, the total moles of ethylene oxide is preferably less than 5, and more preferably less than 2.

The fatty acid esters or fatty ethers of polyhydric alcohols can be alkoxylated, preferably ethoxylated and/or propoxylated, by conventional techniques. Alkoxylating compounds are preferably selected from the group consisting of ethylene oxide, propylene oxide, and mixtures thereof, and similar oxirane compounds.

The compositions of the present invention include one or more fatty acid esters, fatty ethers, alkoxylated fatty acid esters, or alkoxylated fatty ethers at a suitable level to produce the desired result. Such compositions preferably include a total amount of such material of at least 0.01 percent by weight (wt-%), more preferably at least 0.1 wt-%, even more preferably at least 0.25 wt-%, even more preferably at least 0.5 wt-%, and even more preferably at least 1 wt-%, based on the total weight of the “ready to use” or “as used” composition. In a preferred embodiment, they are present in a total amount of no greater than 20 wt-%, more preferably no greater than 15 wt-%, even more preferably no greater than 10 wt-%, and even more preferably no greater than 5 wt-%, based on the “ready to use” or “as used” composition. Certain compositions may be higher in concentration if they are intended to be diluted prior to use.

Preferred compositions of the present invention that include one or more fatty acid monoesters, fatty monoethers, or alkoxylated derivatives thereof can also include a small amount of a di- or tri-fatty acid ester (i.e., a fatty acid di- or tri-ester), a di- or tri-fatty ether (i.e., a fatty di- or tri-ether), or alkoxylated derivative thereof. Preferably, such components are present in an amount of no more than 50 wt-%, more preferably no more than 40 wt-%, even more preferably no more than 25 wt-%, even more preferably no more than 15 wt-%, even more preferably no more than 10 wt-%, even more preferably no more than 7 wt-%, even more preferably no more than 6 wt-%, and even more preferably no more than 5 wt-%, based on the total weight of the antimicrobial lipid component. For example, for monoesters, monoethers, or alkoxylated derivatives of glycerin, preferably there is no more than 15 wt-%, more preferably no more than 10 wt-%, even more preferably no more than 7 wt-%, even more preferably no more than 6 wt-%, and even more preferably no more than 5 wt-% of a diester, diether, triester, triether, or alkoxylated derivatives thereof present, based on the total weight of the antimicrobial lipid components present in the composition. However, as will be explained in greater detail below, higher concentrations of di- and tri-esters may be tolerated in the raw material if the formulation initially includes free glycerin because of transesterification reactions.

Although in some situations it is desirable to avoid di- or tri-esters as a component of the starting materials, it is possible to use relatively pure tri-esters in the preparation of certain compositions of the present invention (for example, as a hydrophobic component) and have effective antimicrobial activity.

The antimicrobial lipid component of the present invention is preferably not reactive with other ingredients or components within the composition and therefore would not be either totally or partially modified or consumed within the composition. Such reactivity could significant impact the antimicrobial activity of the antimicrobial lipid component and of the entire composition.

To achieve rapid antimicrobial activity, formulations may incorporate one or more antimicrobial lipids in the composition approaching, or preferably exceeding, the solubility limit in the hydrophobic phase. While not intended to be bound by theory, it appears that antimicrobial lipids that preferably partition into the hydrophobic component are not readily available to kill microorganisms which are in or associated with an aqueous phase in or on the tissue. In most compositions, the antimicrobial lipid is preferably incorporated in at least 60%, preferably, at least 75%, more preferably, at least 100%, and most preferably, at least 120%, of the solubility limit of the hydrophobic component at 23° C. This is conveniently determined by making the formulation without the antimicrobial lipid, separating the phases (e.g., by centrifugation or other suitable separation technique) and determining the solubility limit by addition of progressively greater levels of the antimicrobial lipid until precipitation occurs. One skilled in the art will realize that creation of supersaturated solutions must be avoided for an accurate determination.

Enhancer Component

Compositions of the present invention preferably include an enhancer (preferably a synergist) to enhance the antimicrobial activity especially against Gram negative bacteria, such as E. coli and Psuedomonas sp. The chosen enhancer preferably affects the cell envelope of the bacteria. While not bound by theory, it is presently believed that the enhancer functions by allowing the antimicrobial lipid to more easily enter the cell cytoplasm and/or by facilitating disruption of the cell envelope. The enhancer component may include an alpha-hydroxy acid, a beta-hydroxy acid, other carboxylic acids, a phenolic compound (such as certain antioxidants and parabens), a monohydroxy alcohol, a chelating agent, or a glycol ether (i.e., ether glycol). Various combinations of enhancers can be used if desired.

The alpha-hydroxy acid, beta-hydroxy acid, and other carboxylic acid enhancers are preferably present in their protonated, free acid form. It is not necessary for all of the acidic enhancers to be present in the free acid form; however, the preferred concentrations listed below refer to the amount present in the free acid form. Additional, non-alpha hydroxy acid, beta-hydroxy acid or other carboxylic acid enhancers, may be added in order to acidify the formulation or buffer it at a pH to maintain antimicrobial activity. Furthermore, the chelator enhancers that include carboxylic acid groups are preferably present with at least one, and more preferably at least two, carboxylic acid groups in their free acid form. The concentrations given below assume this to be the case.

One or more enhancers may be used in the compositions of the present invention at a suitable level to produce the desired result. In a preferred embodiment, they are present in a total amount greater than 0.01 wt-%, more preferably in an amount greater than 0.1 wt-%, even more preferably in an amount greater than 0.2 wt-%, even more preferably in an amount greater than 0.25 wt-%, and most preferably in an amount greater than 0.4 wt-% based on the total weight of the ready to use composition. In a preferred embodiment, they are present in a total amount of no greater than 20 wt-%, based on the total weight of the ready to use composition. Such concentrations typically apply to alpha-hydroxy acids, beta-hydroxy acids, other carboxylic acids, chelating agents, phenolics, ether glycols, and (C5-C10)monohydroxy alcohols. Generally, higher concentrations are needed for (C1-C4)monohydroxy alcohols, as described in greater detail below.

The alpha-hydroxy acid, beta-hydroxy acid, and other carboxylic acid enhancers, as well as chelators that include carboxylic acid groups, are preferably present in a concentration of no greater than 100 milliMoles per 100 grams of formulated composition. In most embodiments, alpha-hydroxy acid, beta-hydroxy acid, and other carboxylic acid enhancers, as well as chelators that include carboxylic acid groups, are preferably present in a concentration of no greater than 75 milliMoles per 100 grams, more preferably no greater than 50 milliMoles per 100 grams, and most preferably no greater than 25 milliMoles per 100 grams of formulated composition.

The total concentration of the enhancer component relative to the total concentration of the antimicrobial lipid component is preferably within a range of 10:1 to 1:300, and more preferably 5:1 to 1:10, on a weight basis.

An additional consideration when using an enhancer is the solubility and physical stability in the compositions. Many of the enhancers discussed herein are insoluble in hydrophobic components.

Alternatively, the enhancer may be present in excess of the solubility limit provided that the composition is physically stable. This may be achieved by utilizing a sufficiently viscous composition that stratification (e.g., settling or creaming) of the antimicrobial lipid does not appreciably occur.

Alpha-hydroxy Acids

An alpha-hydroxy acid is typically a compound represented by the formula:
R⁵(CR⁶OH)ₙCOOH
wherein: R⁵ and R⁶ are each independently H, a (C1-C8)alkyl group (straight, branched, or cyclic group), a (C6-C12)aryl group, a (C6-C12)aralkyl group, or a (C6-C12)alkaryl group (wherein the alkyl group of the aralkyl or alkaryl is straight, branched, or cyclic), wherein R⁵ and R⁶ may be optionally substituted with one or more carboxylic acid groups; and n 1-3, preferably, n=1-2.

Exemplary alpha-hydroxy acids include, but are not limited to, lactic acid, malic acid, citric acid, 2-hydroxybutanoic acid, mandelic acid, gluconic acid, glycolic acid, tartaric acid, alpha-hydroxyoctanoic acid, and alpha-hydroxycaprylic acid, as well as derivatives thereof (e.g., compounds substituted with hydroxyls, phenyl groups, hydroxyphenyl groups, alkyl groups, halogens, as well as combinations thereof). Preferred alpha-hydroxy acids include lactic acid, malic acid, and mandelic acid. These acids may be in D, L, or DL form and may be present as free acid, lactone, or partial salts thereof. All such forms are encompassed by the term “acid.” Preferably, the acids are present in the free acid form. In certain preferred embodiments, the alpha-hydroxy acids useful in the compositions of the present invention are selected from the group consisting of lactic acid, mandelic acid, and malic acid, and mixtures thereof. Other suitable alpha-hydroxy acids are described in U.S. Pat. No. 5,665,776 (Yu).

One or more alpha-hydroxy acids may be used in the compositions of the present invention at a suitable level to produce the desired result. In a preferred embodiment, they are present in a total amount of at least 0.25 wt-%, more preferably, at least 0.5 wt-%, and even more preferably, at least 1 wt-%, based on the total weight of the ready to use composition. In a preferred embodiment, they are present in a total amount of no greater than 10 wt-%, more preferably, no greater than 5 wt-%, and even more preferably, no greater than 3 wt-%, based on the total weight of the ready to use composition. Higher concentrations may become irritating.

The ratio of alpha-hydroxy acid enhancer to total antimicrobial lipid component is preferably at most 10:1, more preferably at most 5:1, and even more preferably at most 1:1. The ratio of alpha-hydroxy acid enhancer to total antimicrobial lipid component is preferably at least 1:20, more preferably at least 1:12, and even more preferably at least 1:5. Preferably the ratio of alphahydroxy acid enhancer to total antimicrobial lipid component is within a range of 1:12 to 1:1.

Beta-hydroxy Acids

A beta-hydroxy acid is typically a compound represented by the formula: 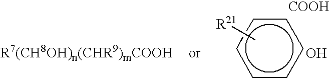
wherein: R⁷, R⁸, and R⁹ are each independently H, a (C1-C8)alkyl group (saturated straight, branched, or cyclic group), a (C6-C12)aryl group, a (C6-C12)aralkyl group, or a (C6-C12)alkaryl group (wherein the alkyl group of the aralkyl or alkaryl is straight, branched, or cyclic), wherein R⁷ and R⁸ may be optionally substituted with one or more carboxylic acid groups; m=0 or 1; n=1-3 (preferably, n=1-2); and R²¹ is H, (C1-C4)alkyl or a halogen.

Exemplary beta-hydroxy acids include, but are not limited to, salicylic acid, beta-hydroxybutanoic acid, tropic acid, 4-aminosalicylic acid, and trethocanic acid. In certain preferred embodiments, the beta-hydroxy acids useful in the compositions of the present invention are selected from the group consisting of salicylic acid, beta-hydroxybutanoic acid, and mixtures thereof. Other suitable beta-hydroxy acids are described in U.S. Pat. No. 5,665,776 (Yu).

One or more beta-hydroxy acids may be used in the compositions of the present invention at a suitable level to produce the desired result. In a preferred embodiment, they are present in a total amount of at least 0.1 wt-%, more preferably at least 0.25 wt-%, and even more preferably at least 0.5 wt-%, based on the total weight of the ready to use composition. In a preferred embodiment, they are present in a total amount of no greater than 10 wt-%, more preferably no greater than 5 wt-%, and even more preferably no greater than 3 wt-%, based on the total weight of the ready to use composition. Higher concentrations may become irritating.

The ratio of beta-hydroxy acid enhancer to total antimicrobial lipid component is preferably at most 10:1, more preferably at most 5:1, and even more preferably at most 1:1. The ratio of beta-hydroxy acid enhancer to total antimicrobial lipid component is preferably at least 1:20, more preferably at least 1:15, and even more preferably at least 1:10. Preferably the ratio of beta-hydroxy acid enhancer to total antimicrobial lipid component is within a range of 1:15 to 1:1.

In systems with low concentrations of water, or that are essentially free of water, transesterification may be the principle route of loss of the fatty acid monoester and alkoxylated derivatives of these active ingredients and loss of carboxylic acid containing enhancers may occur due to esterification. Thus, certain alpha-hydroxy acids (AHA) and beta-hydroxy acids (BHA) are particularly preferred since these are believed to be less likely to transesterify the ester antimicrobial lipid or other esters by reaction of the hydroxyl group of the AHA or BHA. For example, salicylic acid may be particularly preferred in certain formulations since the phenolic hydroxyl group is much more acidic than an aliphatic hydroxyl group and thus much less likely to react. Other particularly preferred compounds in anhydrous or low-water content formulations include lactic, mandelic, malic, citric, tartaric, and glycolic acid. Benzoic acid and substituted benzoic acids that do not include a hydroxyl group, while not hydroxy acids, are also preferred due to a reduced tendency to form ester groups.

Other Carboxylic Acids

Carboxylic acids other than alpha- and beta-carboxylic acids are suitable for use in the enhancer component. These include alkyl, aryl, aralkyl, or alkaryl carboxylic acids typically having equal to or less than 16, and often equal to or less than 12 carbon atoms.

A preferred class of these can be represented by the following formula:
R¹⁰(CR¹¹₂)ₙCOOH
wherein: R¹⁰ and R¹¹ are each independently H, a (C1-C4)alkyl group (which can be a straight, branched, or cyclic group), a (C6-C12)aryl group, a (C6-C16) group containing both aryl groups and alkyl groups (which can be a straight, branched, or cyclic group), wherein R¹⁰ and R¹¹ may be optionally substituted with one or more carboxylic acid groups; and n=0-3, preferably, n=0-2. Preferably, the carboxylic acid is a (C1-C4)alkyl carboxylic acid, a (C6-C12)aralkyl carboxylic acid, or a (C6-C16)alkaryl carboxylic acid.

Exemplary acids include, but are not limited to, acetic acid, propionic acid, benzoic acid, benzylic acid, nonylbenzoic acid, p-hydroxybenzoic acid, retinoic acid, and the like. Particularly preferred is benzoic acid.

One or more carboxylic acids (other than alpha- or beta-hydroxy acids) may be used in the compositions of the present invention at a suitable level to produce the desired result. In a preferred embodiment, they are present in a total amount of at least 0.1 wt-%, more preferably at least 0.25 wt-%, even more preferably at least 0.5 wt-%, and most preferably at least 1 wt-%, based on the ready to use concentration composition. In a preferred embodiment, they are present in a total amount of no greater than 10 wt-%, more preferably no greater than 5 wt-%, and even more preferably no greater than 3 wt-%, based on the ready to use composition.

The ratio of the total concentration of carboxylic acids (other than alpha- or beta-hydroxy acids) to the total concentration of the antimicrobial lipid component is preferably within a range of 10:1 to 1:100, and more preferably 2:1 to 1:10, on a weight basis.

Chelators

A chelating agent (i.e., chelator) is typically an organic compound capable of multiple coordination sites with a metal ion in solution. Typically these chelating agents are polyanionic compounds and coordinate best with polyvalent metal ions. Exemplary chelating agents include, but are not limited to, ethylene diamine tetraacetic acid (EDTA) and salts thereof (e.g., EDTA(Na)₂, EDTA(Na)₄, EDTA(Ca), EDTA(K)₂), sodium acid pyrophosphate, acidic sodium hexametaphosphate, adipic acid, succinic acid, polyphosphoric acid, sodium acid pyrophosphate, sodium hexametaphosphate, acidified sodium hexametaphosphate, nitrilotris(methylenephosphonic acid), diethylenetriaminepentaacetic acid, ethylenebis(oxyethylenenitrilo)tetraacetic acid, glycolether diaminetetraacetic acid, ethyleneglycol-O, O′bis(2-aminoethyl)-N,N,N′,N′-tetraacetic acid (EGTA), N-(2-hydroxyethyl)ethylenediamine-N,N′,N′-triacetic acid trisodium salt (HETA), polyethylene glycol diaminetetraacetic acid, 1-hydroxyethylene, 1,1-diphosphonic acid (HEDP), and diethylenetriaminepenta-(methylenephosphonic acid). Any of these chelating agents may also be used in their partial or complete salt form. Certain carboxylic acids, particularly the alpha-hydroxy acids and beta-hydroxy acids, can also function as chelators, e.g., malic acid, ctiric, and tartaric acid.

Also included as chelators are compounds highly specific for binding ferrous and/or ferric ion such as siderophores, and iron binding proteins. Iron binding proteins include, for example, lactoferrin, and transferrin. Siderophores include, for example, enterochelin, enterobactin, vibriobactin, anguibactin, pyochelin, pyoverdin, and aerobactin.

In certain preferred embodiments, the chelating agents useful in the compositions of the present invention include those selected from the group consisting of ethylenediaminetetraacetic acid and salts thereof, succinic acid, and mixtures thereof. Preferably, either the free acid or the mono- or di-salt form of EDTA is used.

One or more chelating agents may be used in the compositions of the present invention at a suitable level to produce the desired result. In a preferred embodiment, they are present in a total amount of at least 0.01 wt-%, more preferably at least 0.05 wt-%, even more preferably at least 0.1 wt-%, and even more preferably at least 1 wt-%, based on the weight of the ready to use composition. In a preferred embodiment, they are present in a total amount of no greater than 10 wt-%, more preferably no greater than 5 wt-%, and even more preferably no greater than 1 wt-%, based on the weight of the ready to use composition.

The ratio of the total concentration of chelating agents (other than alpha- or beta-hydroxy acids) to the total concentration of the antimicrobial lipid component is preferably within a range of 10:1 to 1:100, and more preferably 1:1 to 1:10, on a weight basis.

Phenolic Compounds

A phenolic compound enhancer (i.e., a phenol or a phenol derivative) is typically a compound having the following general structure (including at least one group bonded to the ring through an oxygen): 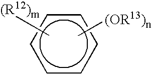
wherein: m is 0 to 3 (especially 1 to 3), n is 1 to 3 (especially 1 to 2), each R¹² independently is alkyl or alkenyl of up to 12 carbon atoms (especially up to 8 carbon atoms) optionally substituted with 0 in or on the chain (e.g., as a carbonyl group) or OH on the chain, and each R¹³ independently is H or alkyl or alkenyl of up to 8 carbon atoms (especially up to 6 carbon atoms) optionally substituted with 0 in or on the chain (e.g., as a carbonyl group) or OH on the chain, but where R¹³ is H, n preferably is 1 or 2.

Examples of phenolic enhancers include, but are not limited to, butylated hydroxy anisole, e.g., 3(2)-tert-butyl-4-methoxyphenol (BHA), 2,6-di-tert-butyl-4-methylphenol (BHT), 3,5-di-tert-butyl-4-hydroxybenzylphenol, 2,6-di-tert-4-hexylphenol, 2,6-di-tert-4-octylphenol, 2,6-di-tert-4-decylphenol, 2,6-di-tert-butyl-4-ethylphenol, 2,6-di-tert-4-butylphenol, 2,5-di-tert-butylphenol, 3,5-di-tert-butylphenol, 4,6-di-tert-butyl-resorcinol, methyl paraben (4-hydroxybenzoic acid methyl ester), ethyl paraben, propyl paraben, butyl paraben, as well as combinations thereof. A preferred group of the phenolic compounds is the phenol species having the general structure shown above where R¹³═H and where R¹² is alkyl or alkenyl of up to 8 carbon atoms, and n is 1, 2, or 3, especially where at least one R¹² is butyl and particularly tert-butyl, and especially the non-toxic members thereof. Some of the preferred phenolic synergists are BHA, BHT, methyl paraben, ethyl paraben, propyl paraben, and butyl paraben as well as combinations of these.

One or more phenolic compounds may be used in the compositions of the present invention at a suitable level to produce the desired result. The concentrations of the phenolic compounds in medical-grade compositions may vary widely, but as little as 0.001 wt-%, based on the total weight of the composition, can be effective when the above-described esters are present within the above-noted ranges. In a preferred embodiment, they are present in a total amount of at least 0.01 wt-%, more preferably at least 0.10 wt-%, and even more preferably at least 0.25 wt-%, based on the ready to use composition. In a preferred embodiment, they are present in a total amount of no greater than 8 wt-%, more preferably no greater than 4 wt-%, and even more preferably no greater than 2 wt-%, based on the ready to use composition.

It is preferred that the ratio of the total phenolic concentration to the total concentration of the antimicrobial lipid component be within a range of 10:1 to 1:300, and more preferably within a range of 1:1 to 1:10, on a weight basis.

The above-noted concentrations of the phenolics are normally observed unless concentrated formulations for subsequent dilution are intended. On the other hand, the minimum concentration of the phenolics and the antimicrobial lipid components to provide an antimicrobial effect will vary with the particular application.

Monohydroxy Alcohols

An additional enhancer class includes monohydroxy alcohols having 1-10 carbon atoms. This includes the lower (i.e., C1-C4) monohydroxy alcohols (e.g., methanol, ethanol, isopropanol, and butanol) as well as longer chain (i.e., C5-C10) monohydroxy alcohols (e.g., isobutanol, t-butanol, octanol, and decanol). Other useful alcohols include phenoxyethanol, benzyl alcohol, and menthol. In certain preferred embodiments, the alcohols useful in the compositions of the present invention are selected from the group consisting of methanol, ethanol, isopropyl alcohol, and mixtures thereof.

One or more alcohols may be used in the compositions of the present invention at a suitable level to produce the desired result. In a preferred embodiment, the short chain (i.e., C1-C4) alcohols are present in a total amount of at least 10 wt-%, even more preferably at least 15 wt-%, even more preferably at least 20 wt-%, and even more preferably at least 25 wt-%, based on the total weight of the ready to use composition.

In a preferred embodiment, the (C1-C4)alcohols are present in a total amount of no greater than 90 wt-%, more preferably no greater than 70 wt-%, even more preferably no greater than 60 wt-%, and even more preferably no greater than 50 wt-%, based on the total weight of the ready to use composition.

For certain applications, lower alcohols may not be preferred due to the strong odor and potential for stinging and irritation. This can occur especially at higher levels. In applications where stinging or burning is a concern, the concentration of (C1-C4)alcohols is preferably less than 20 wt-%, more preferably less than 15 wt-%.

In another preferred embodiment longer chain (i.e., C5-C10)alcohols are present in a total amount of at least 0.1 wt-%, more preferably at least 0.25 wt-%, and even more preferably at least 0.5 wt-%, and most preferably at least 1.0%, based on the ready to use composition. In a preferred embodiment, the (C5-C10)alcohols are present in a total amount of no greater than 10 wt-%, more preferably no greater than 5 wt-%, and even more preferably no greater than 2 wt-%, based on the total weight of the ready to use composition.

Ether glycols

An additional enhancer class includes ether glycols. Exemplary ether glycols include those of the formula:
R′—O—(CH₂CHR″O)ₙ(CH₂CHR″O)H
wherein R′═H, a (C1-C8)alkyl, a (C6-C12)aryl group, a (C6-C12)aralkyl group, or a (C6-C12)alkaryl group; and each R″ is independently=H, methyl, or ethyl; and n=0-5, preferably 1-3. Examples include 2-phenoxyethanol, dipropylene glycol, triethylene glycol, the line of products available under the trade designation DOWANOL DB (di(ethylene glycol) butyl ether), DOWANOL DPM (di(propylene glycol)monomethyl ether), and DOWANOL TPnB (tri(propylene glycol) monobutyl ether), as well as many others available from Dow Chemical, Midland, Mich.

One or more ether glycols may be used in the compositions of the present invention at a suitable level to produce the desired result. In a preferred embodiment, they are present in a total amount of at least 0.01 wt-%, based on the total weight of the ready to use composition. In a preferred embodiment, they are present in a total amount of no greater than 20 wt-%, based on the total weight of the ready to use composition.

Surfactants

Compositions of the present invention can optionally include one or more surfactants. In some embodiments, the presence of a surfactant may be used to emulsify the composition and to help wet the surface and/or to aid in contacting the microorganisms. As used herein the term “surfactant” means an amphiphile (a molecule possessing both polar and nonpolar regions which are covalently bound) capable of reducing the surface tension of water and/or the interfacial tension between water and an immiscible liquid. The term is meant to include soaps, detergents, emulsifiers, surface active agents, and the like. The surfactant can be cationic, anionic, nonionic, or amphoteric. This includes a wide variety of conventional surfactants. Combinations of various surfactants can be used if desired.

Certain ethoxylated surfactants can reduce or eliminate the antimicrobial efficacy of the antimicrobial lipid component. The exact mechanism of this is not known and not all ethoxylated surfactants display this negative effect. For example, poloxamer (polyethylene oxide/polypropylene oxide) surfactants have been shown to be compatible with the antimicrobial lipid component, but ethoxylated sorbitan fatty acid esters such as those sold under the trade name TWEEN by ICI have not been compatible. It should be noted that these are broad generalizations and the activity could be formulation dependent.

It should be noted that certain antimicrobial lipds are amphiphiles and may be surface active. For example, certain antimicrobial alkyl monoglycerides described herein are surface active. For certain embodiments of the invention, the antimicrobial lipid component is considered distinct from a “surfactant” component.

Preferred surfactants are those that have an HLB (i.e., hydrophile to lipophile balance) of at least 4 and more preferably at least 8. Even more preferred surfactants have an HLB of at least 12. Most preferred surfactants have an HLB of at least 15; however, lower HLB surfactants are still useful in compositions described herein.

Examples of the various classes of surfactants are described below. In certain preferred embodiments, the surfactants useful in the compositions of the present invention are selected from the group consisting of sulfonates, sulfates, phosphonates, phosphates, poloxamer (polyethylene oxide/polypropylene oxide block copolymers), cationic surfactants, and mixtures thereof. In certain more preferred embodiments, the surfactants useful in the compositions of the present invention are selected from the group consisting of sulfonates, sulfates, phosphates, and mixtures thereof.

One or more surfactants may be used in the compositions of the present invention at a suitable level to produce the desired result. In a preferred embodiment, they are present in a total amount of at least 0.1 wt-%, more preferably at least 0.5 wt-%, and even more preferably at least 1.0 wt-%, based on the total weight of the ready to use composition.

Surfactants may be present in a total amount of no greater than 10 wt-%, more preferably no greater than 5 wt-%, even more preferably no greater than 3 wt-%, and even more preferably no greater than 2 wt-%, based on the total weight of the ready to use composition. The ratio of the total concentration of surfactant to the total concentration of the antimicrobial lipid component is preferably within a range of 5:1 to 1:100, more preferably 3:1 to 1:10, and most preferably 2:1 to 1:3, on a weight basis.

Cationic Surfactants

Exemplary cationic surfactants include, but are not limited to, salts of optionally polyoxyalkylenated primary, secondary, or tertiary fatty amines; quaternary ammonium salts such as tetraalkylammonium, alkylamidoalkyltrialkylammonium, trialkylbenzylammonium, trialkylhydroxyalkylammonium, or alkylpyridinium halides (preferably chlorides or bromides) as well as other anionic counterions, such as but not limited to, alkyl sulfates, such as but not limited to, methosulfate and ethosulfate; imidazoline derivatives; amine oxides of a cationic nature (e.g., at an acidic pH); and mixtures thereof.

In certain embodiments, the cationic surfactants useful in the compositions of the present invention are selected from the group consisting of tetralkyl ammonium, trialkylbenzylammonium, and alkylpyridinium halides as well as other anionic counterions, such as but not limited to, C1-C4 alkyl sulfates, such as but not limited to, methosulfate and ethosulfate, and mixtures thereof.

Amine Oxide Surfactants

Amine oxide surfactants, which can be cationic or nonionic depending on the pH (e.g., cationic at lower pH and nonionic at higher pH). Amine oxide surfactants including alkyl and alkylamidoalkyldialkylamine oxides of the following formula:
(R¹⁴)₃—N→O
wherein R¹⁴ is a (C1-C30)alkyl group (preferably a (C1-C14)alkyl group) or a (C6-C18)aralklyl or alkaryl group, wherein any of these groups can be optionally substituted in or on the chain by N—, O—, or S-containing groups such as amide, ester, hydroxyl, and the like. Each R¹⁴ may be the same or different provided at least one R¹⁴ group includes at least eight carbons. Optionally, the R¹⁴ groups can be joined to form a heterocyclic ring with the nitrogen to form surfactants such as amine oxides of alkyl morpholine, alkyl piperazine, and the like. Preferably two R¹⁴ groups are methyl and one R¹⁴ group is a (C12-C16)alkyl or alkylamidopropyl group. Examples of amine oxide surfactants include those commercially available under the trade designations AMMONYX LO, LMDO, and CO, which are lauryldimethylamine oxide, laurylamidopropyldimethylamine oxide, and cetyl amine oxide, all from Stepan Company of Northfield, Ill.
Anionic Surfactants

Exemplary anionic surfactants include, but are not limited to, sarcosinates, glutamates, alkyl sulfates, sodium or potassium alkyleth sulfates, ammonium alkyleth sulfates, ammonium laureth-n-sulfates, laureth-n-sulfates, isethionates, glycerylether sulfonates, sulfosuccinates, alkylglyceryl ether sulfonates, alkyl phosphates, aralkyl phosphates, alkylphosphonates, and aralkylphosphonates. These anionic surfactants may have a metal or organic ammonium counterion. In certain preferred embodiments, the anionic surfactants useful in the compositions of the present invention are selected from the group consisting of:
1. Sulfonates and Sulfates. Suitable anionic surfactants include sulfonates and sulfates such as alkyl sulfates, alkylether sulfates, alkyl sulfonates, alkylether sulfonates, alkylbenzene sufonates, alkylbenzene ether sulfates, alkylsulfoacetates, secondary alkane sulfonates, secondary alkylsulfates, and the like. Many of these can be represented by the formulas:
   R¹⁴—(OCH₂CH₂)ₙ(OCH(CH₃)CH₂)ₚ-(Ph)ₐ-(OCH₂CH₂)ₘ—(O)_{b}—SO₃⁻M⁺
   and
   R¹⁴—CH[SO₃-M⁺]-R¹⁵
   wherein: a and b=0 or 1; n, p, and m=0-100 (preferably 0-20, and more preferably 0-10); R¹⁴ is defined as above provided at least one R¹⁴ or R¹⁵ is at least C8; R¹⁵ is a (C1-C12)alkyl group (saturated straight, branched, or cyclic group) that may be optionally substituted by N, O, or S atoms or hydroxyl, carboxyl, amide, or amine groups; Ph=phenyl; and M is a cationic counterion such as H, Na, K, Li, ammonium, or a protonated tertiary amine such as triethanolamine or a quaternary ammonium group.

In the formula above, the ethylene oxide groups (i.e., the “n” and “m” groups) and propylene oxide groups (i.e., the “p” groups) can occur in reverse order as well as in a random, sequential, or block arrangement. Preferably for this class, R¹⁴ includes an alkylamide group such as R¹⁶—C(O)N(CH₃)CH₂CH₂— as well as ester groups such as —OC(O)—CH₂— wherein R¹⁶ is a (C8-C22)alkyl group (branched, straight, or cyclic group). Examples include, but are not limited to: alkyl ether sulfonates such as lauryl ether sulfates such as POLYSTEP B12 (n=3-4, M=sodium) and B22 (n=12, M=ammonium) available from Stepan Company, Northfield, Ill. and sodium methyl taurate (available under the trade designation NIKKOL CMT30 from Nikko Chemicals Co., Tokyo, Japan); secondary alkane sulfonates such as Hostapur SAS which is a Sodium (C14-C17)secondary alkane sulfonates (alpha-olefin sulfonates) available from Clariant Corp., Charlotte, N.C.; methyl-2-sulfoalkyl esters such as sodium methyl-2-sulfo(C12-16)ester and disodium 2-sulfo(C12-C16)fatty acid available from Stepan Company under the trade designation ALPHASTEP PC-48; alkylsulfoacetates and alkylsulfosuccinates available as sodium laurylsulfoacetate (under the trade designation LANTHANOL LAL) and disodiumlaurethsulfosuccinate (STEPANMILD SL3), both from Stepan Company; alkylsulfates such as ammoniumlauryl sulfate commercially available under the trade designation STEPANOL AM from Stepan Company; dialkylsulfosuccinates such as dioctylsodiumsulfosuccinate available as Aerosol OT from Cytec Industries. Hydrotropes such as DOWFAX hydrotrope from Dow chemical or other diphenyl oxide surfactants may also be used.
2. Phosphates and Phosphonates. Suitable anionic surfactants also include phosphates such as alkyl phosphates, alkylether phosphates, aralkylphosphates, and aralkylether phosphates. Many may be represented by the formula:
   [R¹⁴-(Ph)ₐ-O(CH₂CH₂O)ₙ(CH₂CH(CH₃)O)ₚ]_{q}—P(O)[O⁻M⁺]ᵣ
   wherein: Ph, R¹⁴, a, n, p, and M are defined above; r is 0-2; and q=1-3; with the proviso that when q=1, r=2, and when q=2, r=1, and when q=3, r=0. As above, the ethyl oxide groups (i.e., the “n” groups) and propylene oxide groups (i.e., the “p” groups) can occur in reverse order as well as in a random, sequential, or block arrangement. Examples include a mixture of mono-, di- and tri-(alkyltetraglycolether)-o-phosphoric acid esters generally referred to as trilaureth-4-phosphate commercially available under the trade designation HOSTAPHAT 340KL from Clariant Corp., Charlotte, N.C., as well as PPG-5 ceteth 10 phosphate available under the trade designation CRODAPHOS SG from Croda Inc., Parsipanny, N.J., and mixtures thereof.
   Amphoteric Surfactants

Surfactants of the amphoteric type include surfactants having tertiary amine groups, which may be protonated, as well as quaternary amine containing zwitterionic surfactants. Such surfactants include:

1. Ammonium Carboxylate Amphoterics. This class of surfactants can be represented by the following formula:
R¹⁷—(C(O)—NH)ₐ—R¹⁸—N⁺(R¹⁹)₂—R²⁰—COO⁻
wherein: a=0 or 1; R¹⁷ is a (C7-C21)alkyl group (saturated straight, branched, or cyclic group), a (C6-C22)aryl group, or a (C6-C22)aralkyl or alkaryl group (saturated straight, branched, or cyclic alkyl group), wherein R¹⁷ may be optionally substituted with one or more N, O, or S atoms, or one or more hydroxyl, carboxyl, amide, or amine groups; R¹⁹ is H or a (C1-C8)alkyl group (saturated straight, branched, or cyclic group), wherein R¹⁹ may be optionally substituted with one or more N, O, or S atoms, or one or more hydroxyl, carboxyl, amine groups, a (C6-C9)aryl group, or a (C6-C9)aralkyl or alkaryl group; and R¹⁸ and R²⁰ are each independently a (C1-C10)alkylene group that may be the same or different and may be optionally substituted with one or more N, O, or S atoms, or one or more hydroxyl or amine groups.

In other embodiments, in the formula above, R¹⁷ is a (C1-C18)alkyl group, R¹⁹ is a (C1-C2)alkyl group preferably substituted with a methyl or benzyl group and most preferably with a methyl group. When R¹⁹ is H it is understood that the surfactant at higher pH values could exist as a tertiary amine with a cationic counterion such as Na, K, Li, or a quaternary amine group.

Examples of such amphoteric surfactants include, but are not limited to: certain betaines such as cocobetaine and cocamidopropyl betaine (commercially available under the trade designations MACKAM CB-35 and MACKAM L from McIntyre Group Ltd., University Park, Ill.); monoacetates such as sodium lauroamphoacetate; diacetates such as disodium lauroamphoacetate; and amino- and alkylamino-propionates such as lauraminopropionic acid (commercially available under the trade designations MACKAM 1L, MACKAM 2L, and MACKAM 151L, respectively, from McIntyre Group Ltd.).
2. Ammonium Sulfonate Amphoterics. This class of amphoteric surfactants are often referred to as “sultaines” or “sulfobetaines” and can be represented by the following formula
   R¹⁷—(C(O)—NH)ₐ—R¹⁸—N⁺(R¹⁹)₂—R²⁰—SO₃⁻
   wherein R¹⁷—R²⁰ and “a” are defined above. Examples include cocamidopropylhydroxysultaine (commercially available as MACKAM 50-SB from McIntyre Group Ltd.). The sulfoamphoterics may be preferred over the carboxylate amphoterics since the sulfonate group will remain ionized at much lower pH values.
   Nonionic Surfactants

Exemplary nonionic surfactants include, but are not limited to, alkyl glucosides, alkyl polyglucosides, polyhydroxy fatty acid amides, sucrose esters, esters of fatty acids and polyhydric alcohols, fatty acid alkanolamides, ethoxylated fatty acids, ethoxylated aliphatic acids, ethoxylated fatty alcohols (e.g., octyl phenoxy polyethoxyethanol available under the trade name TRITON X-100 and nonyl phenoxy poly(ethyleneoxy) ethanol available under the trade name NONIDET P-40, both from Sigma, St. Louis, Mo.), ethoxylated and/or propoxylated aliphatic alcohols (e.g., that available under the trade name BRIJ from ICI, Wilmington, Del.), ethoxylated glycerides, ethoxylated/propoxylated block copolymers such as PLURONIC and TETRONIC surfactants available from BASF, ethoxylated cyclic ether adducts, ethoxylated amide and imidazoline adducts, ethoxylated amine adducts, ethoxylated mercaptan adducts, ethoxylated condensates with alkyl phenols, ethoxylated nitrogen-based hydrophobes, ethoxylated polyoxypropylenes, polymeric silicones, fluorinated surfactants (e.g., those available under the trade names FLUORAD-FS 300 from 3M Company, St. Paul, Minn., and ZONYL from Dupont de Nemours Co., Wilmington, Del.), and polymerizable (reactive) surfactants (e.g., SAM 211 (alkylene polyalkoxy sulfate) surfactant available under the trade name MAZON from PPG Industries, Inc., Pittsburgh, Pa.). In certain preferred embodiments, the nonionic surfactants useful in the compositions of the present invention are selected from the group consisting of Poloxamers such as PLURONIC from BASF, sorbitan fatty acid esters, and mixtures thereof. A particularly preferred nonionic surfactant is P65 poloxamer (polyethylene oxide capped polypropylene oxide having a EO/PO mole ratio of 1 and a molecular weight of approximately 3400) available from BASF Wyandotte Corp., Parsippany, N.J.

Hardenable Component

The hardenable dental compositions of the present invention typically include a hardenable (e.g., polymerizable) component, thereby forming hardenable (e.g., polymerizable) compositions. The hardenable component can include a wide variety of chemistries, such as ethylenically unsaturated compounds (with or without acid functionality), epoxy (oxirane) resins, vinyl ethers, photopolymerization systems, redox cure systems, glass ionomer cements, polyethers, polysiloxanes, and the like. In some embodiments, the compositions can be hardened (e.g., polymerized by conventional photopolymerization and/or chemical polymerization techniques) prior to applying the dental material. In other embodiments, the compositions can be hardened (e.g., polymerized by conventional photopolymerization and/or chemical polymerization techniques) after applying the dental material.

In certain embodiments, the compositions are photopolymerizable, i.e., the compositions contain a photoinitiator (i.e., a photoinitiator system) that upon irradiation with actinic radiation initiates the polymerization (or hardening) of the composition. Such photopolymerizable compositions can be free radically polymerizable or cationically polymerizable. In other embodiments, the compositions are chemically hardenable, i.e., the compositions contain a chemical initiator (i.e., initiator system) that can polymerize, cure, or otherwise harden the composition without dependence on irradiation with actinic radiation. Such chemically hardenable compositions are sometimes referred to as “self-cure” compositions and may include glass ionomer cements (e.g., conventional and resin-modified glass ionomer cements), redox cure systems, and combinations thereof.

Suitable photopolymerizable components that can be used in the dental compositions of the present invention include, for example, epoxy resins (which contain cationically active epoxy groups), vinyl ether resins (which contain cationically active vinyl ether groups), ethylenically unsaturated compounds (which contain free radically active unsaturated groups, e.g., acrylates and methacrylates), and combinations thereof Also suitable are polymerizable materials that contain both a cationically active functional group and a free radically active functional group in a single compound. Examples include epoxy-functional acrylates, epoxy-functional methacrylates, and combinations thereof.

Ethylenically Unsaturated Compounds

The compositions of the present invention may include one or more hardenable components in the form of ethylenically unsaturated compounds with or without acid functionality, thereby forming hardenable compositions.

Suitable hardenable compositions may include hardenable components (e.g., photopolymerizable compounds) that include ethylenically unsaturated compounds (which contain free radically active unsaturated groups). Examples of useful ethylenically unsaturated compounds include acrylic acid esters, methacrylic acid esters, hydroxy-functional acrylic acid esters, hydroxy-functional methacrylic acid esters, and combinations thereof.

The compositions (e.g., photopolymerizable compositions) may include compounds having free radically active functional groups that may include monomers, oligomers, and polymers having one or more ethylenically unsaturated group. Suitable compounds contain at least one ethylenically unsaturated bond and are capable of undergoing addition polymerization. Such free radically polymerizable compounds include mono-, di- or poly-(meth)acrylates (i.e., acrylates and methacrylates) such as, methyl (meth)acrylate, ethyl acrylate, isopropyl methacrylate, n-hexyl acrylate, stearyl acrylate, allyl acrylate, glycerol triacrylate, ethyleneglycol diacrylate, diethyleneglycol diacrylate, triethyleneglycol dimethacrylate, 1,3-propanediol di(meth)acrylate, trimethylolpropane triacrylate, 1,2,4-butanetriol trimethacrylate, 1,4-cyclohexanediol diacrylate, pentaerythritol tetra(meth)acrylate, sorbitol hexacrylate, tetrahydrofurfuryl(meth)acrylate, bis[1-(2-acryloxy)]-p-ethoxyphenyldimethylmethane, bis[1-(3-acryloxy-2-hydroxy)]-p-propoxyphenyldimethylmethane, ethoxylated bisphenolA di(meth)acrylate, and trishydroxyethyl-isocyanurate trimethacrylate; (meth)acrylamides (i.e., acrylamides and methacrylamides) such as (meth)acrylamide, methylene bis-(meth)acrylamide, and diacetone (meth)acrylamide; urethane (meth)acrylates; the bis-(meth)acrylates of polyethylene glycols (preferably of molecular weight 200-500), copolymerizable mixtures of acrylated monomers such as those in U.S. Pat. No. 4,652, 274 (Boettcher et al.), acrylated oligomers such as those of U.S. Pat. No. 4,642,126 (Zador et al.), and poly(ethylenically unsaturated) carbamoyl isocyanurates such as those disclosed in U.S. Pat. No. 4,648,843 (Mitra); and vinyl compounds such as styrene, diallyl phthalate, divinyl succinate, divinyl adipate and divinyl phthalate. Other suitable free radically polymerizable compounds include siloxane-functional (meth)acrylates as disclosed, for example, in WO-00/38619 (Guggenberger et al.), WO-01/92271 (Weinmann et al.), WO-01/07444 (Guggenberger et al.), WO-00/42092 (Guggenberger et al.) and fluoropolymer-functional (meth)acrylates as disclosed, for example, in U.S. Pat. No. 5,076,844 (Fock et al.), U.S. Pat. No. 4,356,296 (Griffith et al.), EP-0373 384 (Wagenknecht et al.), EP-0201 031 (Reiners et al.), and EP-0201 778 (Reiners et al.). Mixtures of two or more free radically polymerizable compounds can be used if desired.

The hardenable component may also contain hydroxyl groups and ethylenically unsaturated groups in a single molecule. Examples of such materials include hydroxyalkyl (meth)acrylates, such as 2-hydroxyethyl (meth)acrylate and 2-hydroxypropyl (meth)acrylate; glycerol mono- or di-(meth)acrylate; trimethylolpropane mono- or di-(meth)acrylate; pentaerythritol mono-, di-, and tri-(meth)acrylate; sorbitol mono-, di-, tri-, tetra-, or penta-(meth)acrylate; and 2,2-bis[4-(2-hydroxy-3-ethacryloxypropoxy)phenyl]propane (bisGMA). Suitable ethylenically unsaturated compounds are also available from a wide variety of commercial sources, such as Sigma-Aldrich, St. Louis. Mixtures of ethylenically unsaturated compounds can be used if desired.

In certain embodiments hardenable components include PEGDMA (polyethyleneglycol dimethacrylate having a molecular weight of approximately 400), bisGMA, UDMA (urethane dimethacrylate), GDMA (glycerol dimethacrylate), TEGDMA (triethyleneglycol dimethacrylate), bisEMA6 as described in U.S. Pat. No. 6,030,606 (Holmes), and NPGDMA (neopentylglycol dimethacrylate). Various combinations of the hardenable components can be used if desired.

Preferably, compositions of the present invention include at least 5% by weight, more preferably at least 10% by weight, and most preferably at least 15% by weight ethylenically unsaturated compounds, based on the total weight of the unfilled composition. Preferably, compositions of the present invention include at most 95% by weight, more preferably at most 90% by weight, and most preferably at most 80% by weight ethylenically unsaturated compounds, based on the total weight of the unfilled composition.

Preferably, compositions of the present invention include ethylenically unsaturated compounds without acid functionality. Preferably, compositions of the present invention include at least 5% by weight (wt-%), more preferably at least 10% by weight, and most preferably at least 15% by weight ethylenically unsaturated compounds without acid functionality, based on the total weight of the unfilled composition. Preferably, compositions of the present invention include at most 95% by weight, more preferably at most 90% by weight, and most preferably at most 80% by weight ethylenically unsaturated compounds without acid functionality, based on the total weight of the unfilled composition.

Ethylenically Unsaturated Compounds with Acid Functionality

The compositions of the present invention may include one or more hardenable components in the form of ethylenically unsaturated compounds with acid functionality, thereby forming hardenable compositions.

As used herein, ethylenically unsaturated compounds with acid functionality is meant to include monomers, oligomers, and polymers having ethylenic unsaturation and acid and/or acid-precursor functionality. Acid-precursor functionalities include, for example, anhydrides, acid halides, and pyrophosphates. The acid functionality can include carboxylic acid functionality, phosphoric acid functionality, phosphonic acid functionality, sulfonic acid functionality, or combinations thereof.

Ethylenically unsaturated compounds with acid functionality include, for example, α,β-unsaturated acidic compounds such as glycerol phosphate mono(meth)acrylates, glycerol phosphate di(meth)acrylates, hydroxyethyl (meth)acrylate (e.g., HEMA) phosphates, bis((meth)acryloxyethyl) phosphate, ((meth)acryloxypropyl) phosphate, bis((meth)acryloxypropyl) phosphate, bis((meth)acryloxy)propyloxy phosphate, (meth)acryloxyhexyl phosphate, bis((meth)acryloxyhexyl) phosphate, (meth)acryloxyoctyl phosphate, bis((meth)acryloxyoctyl) phosphate, (meth)acryloxydecyl phosphate, bis((meth)acryloxydecyl) phosphate, caprolactone methacrylate phosphate, citric acid di- or tri-methacrylates, poly(meth)acrylated oligomaleic acid, poly(meth)acrylated polymaleic acid, poly(meth)acrylated poly(meth)acrylic acid, poly(meth)acrylated polycarboxyl-polyphosphonic acid, poly(meth)acrylated polychlorophosphoric acid, poly(meth)acrylated polysulfonate, poly(meth)acrylated polyboric acid, and the like, may be used as components in the hardenable component system. Also monomers, oligomers, and polymers of unsaturated carbonic acids such as (meth)acrylic acids, aromatic (meth)acrylated acids (e.g., methacrylated trimellitic acids), and anhydrides thereof can be used. Certain preferred compositions of the present invention include an ethylenically unsaturated compound with acid functionality having at least one P—OH moiety.

Certain of these compounds are obtained, for example, as reaction products between isocyanatoalkyl (meth)acrylates and carboxylic acids. Additional compounds of this type having both acid-functional and ethylenically unsaturated components are described in U.S. Pat. No. 4,872,936 (Engelbrecht) and U.S. Pat. No. 5,130,347 (Mitra). A wide variety of such compounds containing both the ethylenically unsaturated and acid moieties can be used. Mixtures of such compounds can be used if desired.

Additional ethylenically unsaturated compounds with acid functionality include, for example, polymerizable bisphosphonic acids as disclosed for example, in U.S. Pat. Publication No. 2004/0206932 (Abuelyaman et al.); AA:ITA:IEM (copolymer of acrylic acid:itaconic acid with pendent methacrylate made by reacting AA:ITA copolymer with sufficient 2-isocyanatoethyl methacrylate to convert a portion of the acid groups of the copolymer to pendent methacrylate groups as described, for example, in Example 11 of U.S. Pat. No. 5,130,347 (Mitra)); and those recited in U.S. Pat. No. 4,259,075 (Yamauchi et al.), U.S. Pat. No. 4,499,251 (Omura et al.), U.S. Pat. No. 4,537,940 (Omura et al.), U.S. Pat. No. 4,539,382 (Omura et al.), U.S. Pat. No. 5,530,038 (Yamamoto et al.), U.S. Pat. No. 6,458,868 (Okada et al.), and European Pat. Application Publication Nos. EP 712,622 (Tokuyama Corp.) and EP 1,051,961 (Kuraray Co., Ltd.).

Compositions of the present invention can also include compositions that include combinations of ethylenically unsaturated compounds with acid functionality. Preferably the compositions are self-adhesive and are non-aqueous. For example, such compositions can include: a first compound including at least one (meth)acryloxy group and at least one —O—P(O)(OH)ₓ group, wherein x=1 or 2, and wherein the at least one —O—P(O)(OH)ₓ group and the at least one (meth)acryloxy group are linked together by a C1-C4 hydrocarbon group; a second compound including at least one (meth)acryloxy group and at least one —O—P(O)(OH)ₓ group, wherein x=1 or 2, and wherein the at least one —O—P(O)(OH)ₓ group and the at least one (meth)acryloxy group are linked together by a C5-C12 hydrocarbon group; an ethylenically unsaturated compound without acid functionality; an initiator system; and a filler. Such compositions are described, for example, in U.S. Provisional Application Ser. No. 60/600,658 (Luchterhandt et al.), filed on Aug. 11, 2004.

Preferably, the compositions of the present invention include at least 1% by weight, more preferably at least 3% by weight, and most preferably at least 5% by weight ethylenically unsaturated compounds with acid functionality, based on the total weight of the unfilled composition. Preferably, compositions of the present invention include at most 80% by weight, more preferably at most 70% by weight, and most preferably at most 60% by weight ethylenically unsaturated compounds with acid functionality, based on the total weight of the unfilled composition.

Epoxy (Oxirane) or Vinyl Ether Compounds

The hardenable compositions of the present invention may include one or more hardenable components in the form of epoxy (oxirane) compounds (which contain cationically active epoxy groups) or vinyl ether compounds (which contain cationically active vinyl ether groups), thereby forming hardenable compositions.

The epoxy or vinyl ether monomers can be used alone as the hardenable component in a dental composition or in combination with other monomer classes, e.g., ethylenically unsaturated compounds as described herein, and can include as part of their chemical structures aromatic groups, aliphatic groups, cycloaliphatic groups, and combinations thereof.

Examples of epoxy (oxirane) compounds include organic compounds having an oxirane ring that is polymerizable by ring opening. These materials include monomeric epoxy compounds and epoxides of the polymeric type and can be aliphatic, cycloaliphatic, aromatic or heterocyclic. These compounds generally have, on the average, at least 1 polymerizable epoxy group per molecule, in some embodiments at least 1.5, and in other embodiments at least 2 polymerizable epoxy groups per molecule. The polymeric epoxides include linear polymers having terminal epoxy groups (e.g., a diglycidyl ether of a polyoxyalkylene glycol), polymers having skeletal oxirane units (e.g., polybutadiene polyepoxide), and polymers having pendent epoxy groups (e.g., a glycidyl methacrylate polymer or copolymer). The epoxides may be pure compounds or may be mixtures of compounds containing one, two, or more epoxy groups per molecule. The “average” number of epoxy groups per molecule is determined by dividing the total number of epoxy groups in the epoxy-containing material by the total number of epoxy-containing molecules present.

These epoxy-containing materials may vary from low molecular weight monomeric materials to high molecular weight polymers and may vary greatly in the nature of their backbone and substituent groups. Illustrative of permissible substituent groups include halogens, ester groups, ethers, sulfonate groups, siloxane groups, carbosilane groups, nitro groups, phosphate groups, and the like. The molecular weight of the epoxy-containing materials may vary from 58 to 100,000 or more.

Suitable epoxy-containing materials useful as the resin system reactive components in the present invention are listed in U.S. Pat. No. 6,187,836 (Oxman et al.) and U.S. Pat. No. 6,084,004 (Weinmann et al.).

Other suitable epoxy resins useful as the resin system reactive components include those which contain cyclohexene oxide groups such as epoxycyclohexanecarboxylates, typified by 3,4-epoxycyclohexylmethyl-3,4-epoxycyclohexanecarboxylate, 3,4-epoxy-2-methylcyclohexylmethyl-3,4-epoxy-2-methylcyclohexane carboxylate, and bis(3,4-epoxy-6-methylcyclohexyl-methyl) adipate. For a more detailed list of useful epoxides of this nature, reference is made to U.S. Pat. No. 6,245,828 (Weinmann et al.) and U.S. Pat. No. 5,037,861 (Crivello et al.); and U.S. Pat. Publication No. 2003/035899 (Klettke et al.).

Other epoxy resins that may be useful in the compositions of this invention include glycidyl ether monomers. Examples are glycidyl ethers of polyhydric phenols obtained by reacting a polyhydric phenol with an excess of chlorohydrin such as epichlorohydrin (e.g., the diglycidyl ether of 2,2-bis-(2,3-epoxypropoxyphenol)propane). Further examples of epoxides of this type are described in U.S. Pat. No. 3,018,262 (Schroeder), and in “Handbook of Epoxy Resins” by Lee and Neville, McGraw-Hill Book Co., New York (1967).

Other suitable epoxides useful as the resin system reactive components are those that contain silicon, useful examples of which are described in International Pat. Publication No. WO 01/51540 (Klettke et al.).

Additional suitable epoxides useful as the resin system reactive components include octadecylene oxide, epichlorohydrin, styrene oxide, vinyl cyclohexene oxide, glycidol, glycidylmethacrylate, diglycidyl ether of Bisphenol A and other commercially available epoxides, as provided in U.S. Ser. No. 10/719,598 (Oxman et al.; filed Nov. 21, 2003).

Blends of various epoxy-containing materials are also contemplated. Examples of such blends include two or more weight average molecular weight distributions of epoxy-containing compounds, such as low molecular weight (below 200), intermediate molecular weight (200 to 10,000) and higher molecular weight (above 10,000). Alternatively or additionally, the epoxy resin may contain a blend of epoxy-containing materials having different chemical natures, such as aliphatic and aromatic, or functionalities, such as polar and non-polar.

Other types of useful hardenable components having cationically active functional groups include vinyl ethers, oxetanes, spiro-orthocarbonates, spiro-orthoesters, and the like.

If desired, both cationically active and free radically active functional groups may be contained in a single molecule. Such molecules may be obtained, for example, by reacting a di- or poly-epoxide with one or more equivalents of an ethylenically unsaturated carboxylic acid. An example of such a material is the reaction product of UVR-6105 (available from Union Carbide) with one equivalent of methacrylic acid. Commercially available materials having epoxy and free-radically active functionalities include the CYCLOMER series, such as CYCLOMER M-100, M-101, or A-200 available from Daicel Chemical, Japan, and EBECRYL-3605 available from Radcure Specialties, UCB Chemicals, Atlanta, Ga.

The cationically curable components may further include a hydroxyl-containing organic material. Suitable hydroxyl-containing materials may be any organic material having hydroxyl functionality of at least 1, and preferably at least 2. Preferably, the hydroxyl-containing material contains two or more primary or secondary aliphatic hydroxyl groups (i.e., the hydroxyl group is bonded directly to a non-aromatic carbon atom). The hydroxyl groups can be terminally situated, or they can be pendent from a polymer or copolymer. The molecular weight of the hydroxyl-containing organic material can vary from very low (e.g., 32) to very high (e.g., one million or more). Suitable hydroxyl-containing materials can have low molecular weights (i.e., from 32 to 200), intermediate molecular weights (i.e., from 200 to 10,000, or high molecular weights (i.e., above 10,000). As used herein, all molecular weights are weight average molecular weights.

The hydroxyl-containing materials may be non-aromatic in nature or may contain aromatic functionality. The hydroxyl-containing material may optionally contain heteroatoms in the backbone of the molecule, such as nitrogen, oxygen, sulfur, and the like. The hydroxyl-containing material may, for example, be selected from naturally occurring or synthetically prepared cellulosic materials. The hydroxyl-containing material should be substantially free of groups which may be thermally or photolytically unstable; that is, the material should not decompose or liberate volatile components at temperatures below 100° C. or in the presence of actinic light which may be encountered during the desired photopolymerization conditions for the polymerizable compositions.

Suitable hydroxyl-containing materials useful in the present invention are listed in U.S. Pat. No. 6,187,836 (Oxman et al.).

The hardenable component(s) may also contain hydroxyl groups and cationically active functional groups in a single molecule. An example is a single molecule that includes both hydroxyl groups and epoxy groups.

Glass Ionomers

The hardenable compositions of the present invention may include glass ionomer cements such as conventional glass ionomer cements that typically employ as their main ingredients a homopolymer or copolymer of an ethylenically unsaturated carboxylic acid (e.g., poly acrylic acid, copoly (acrylic, itaconic acid), and the like), a fluoroaluminosilicate (“FAS”) glass, water, and a chelating agent such as tartaric acid. Conventional glass ionomers (i.e., glass ionomer cements) typically are supplied in powder/liquid formulations that are mixed just before use. The mixture will undergo self-hardening in the dark due to an ionic reaction between the acidic repeating units of the polycarboxylic acid and cations leached from the glass.

The glass ionomer cements may also include resin-modified glass ionomer (“RMGI”) cements. Like a conventional glass ionomer, an RMGI cement employs an FAS glass. However, the organic portion of an RMGI is different. In one type of RMGI, the polycarboxylic acid is modified to replace or end-cap some of the acidic repeating units with pendent curable groups and a photoinitiator is added to provide a second cure mechanism, e.g., as described in U.S. Pat. No. 5,130,347 (Mitra). Acrylate or methacrylate groups are usually employed as the pendant curable group. In another type of RMGI, the cement includes a polycarboxylic acid, an acrylate or methacrylate-functional monomer and a photoinitiator, e.g., as in Mathis et al., “Properties of a New Glass Ionomer/Composite Resin Hybrid Restorative”, Abstract No. 51, J. Dent Res., 66:113 (1987) and as in U.S. Pat. No. 5,063,257 (Akahane et al.), U.S. Pat. No. 5,520,725 (Kato et al.), U.S. Pat. No. 5,859,089 (Qian), U.S. Pat. No. 5,925,715 (Mitra) and U.S. Pat. No. 5,962,550 (Akahane et al.). In another type of RMGI, the cement may include a polycarboxylic acid, an acrylate or methacrylate-functional monomer, and a redox or other chemical cure system, e.g., as described in U.S. Pat. No. 5,154,762 (Mitra et al.), U.S. Pat. No. 5,520,725 (Kato et al.), and U.S. Pat. No. 5,871,360 (Kato). In another type of RMGI, the cement may include various monomer-containing or resin-containing components as described in U.S. Pat. No. 4,872,936 (Engelbrecht), U.S. Pat. No. 5,227,413 (Mitra), U.S. Pat. No. 5,367,002 (Huang et al.), and U.S. Pat. No. 5,965,632 (Orlowski). RMGI cements are preferably formulated as powder/liquid or paste/paste systems, and contain water as mixed and applied. The compositions are able to harden in the dark due to the ionic reaction between the acidic repeating units of the polycarboxylic acid and cations leached from the glass, and commercial RMGI products typically also cure on exposure of the cement to light from a dental curing lamp. RMGI cements that contain a redox cure system and that can be cured in the dark without the use of actinic radiation are described in U.S. Pat. No. 6,765,038 (Mitra).

Polyethers or Polysiloxanes (i.e., Silicones)

Dental impression materials are typically based on polyether or polysiloxane (i.e. silicone) chemistry. Polyether materials typically consist of a two-part system that includes a base component (e.g., a polyether with ethylene imine rings as terminal groups) and a catalyst (or accelerator) component (e.g., an aryl sulfonate as a cross-linking agent). Polysiloxane materials also typically consist of a two-part system that includes a base component (e.g., a polysiloxane, such as a dimethylpolysiloxane, of low to moderately low molecular weight) and a catalyst (or accelerator) component (e.g., a low to moderately low molecular weight polymer with vinyl terminal groups and chloroplatinic acid catalyst in the case of addition silicones; or a liquid that consists of stannous octanoate suspension and an alkyl silicate in the case of condensation silicones). Both systems also typically contain a filler, a plasticizer, a thickening agent, a coloring agent, or mixtures thereof. Exemplary polyether impression materials include those described in, for example, U.S. Pat. No. 6,127,449 (Bissinger et al.); U.S. Pat. No. 6,395,801 (Bissinger et al.); and U.S. Pat. No. 5,569,691 (Guggenberger et al.). Exemplary polysiloxane impression materials and related polysiloxane chemistry are described in, for example, U.S. Pat. No. 6,121,362 (Wanek et al.) and U.S. Pat. No. 6,566,413 Weinmann et al.), and EP Pat. Publication No. 1 475 069 A (Bissinger et al.).

Examples of commercial polyether and polysiloxane impression materials include, but are not limited to, IMPREGUM Polyether Materials, PERMADYNE Polyether Materials, EXPRESS Vinyl Polysiloxane Materials, DIMENSION Vinyl Polysiloxane Materials, and IMPRINT Vinyl Polysiloxane Materials; all available from 3M ESPE (St. Paul, Minn.). Other exemplary polyether, polysiloxane (silicones), and polysulfide impression materials are discussed in the following reference: Restorative Dental Materials, Tenth Edition, edited by Robert G. Craig and Marcus L. Ward, Mosby-Year Book, Inc., St. Louis, Mo., Chapter 11 (Impression Materials).

Photoinitiator Systems

In certain embodiments, the compositions of the present invention are photopolymerizable, i.e., the compositions contain a photopolymerizable component and a photoinitiator (i.e., a photoinitiator system) that upon irradiation with actinic radiation initiates the polymerization (or hardening) of the composition. Such photopolymerizable compositions can be free radically polymerizable or cationically polymerizable.

Suitable photoinitiators (i.e., photoinitiator systems that include one or more compounds) for polymerizing free radically photopolymerizable compositions include binary and tertiary systems. Typical tertiary photoinitiators include an iodonium salt, a photosensitizer, and an electron donor compound as described in U.S. Pat. No. 5,545,676 (Palazzotto et al.). Preferred iodonium salts are the diaryl iodonium salts, e.g., diphenyliodonium chloride, diphenyliodonium hexafluorophosphate, diphenyliodonium tetrafluoroborate, and tolylcumyliodonium tetrakis(pentafluorophenyl)borate. Preferred photosensitizers are monoketones and diketones that absorb some light within a range of 400 nm to 520 nm (preferably, 450 nm to 500 nm). More preferred compounds are alpha diketones that have some light absorption within a range of 400 nm to 520 nm (even more preferably, 450 to 500 nm). Preferred compounds are camphorquinone, benzil, furil, 3,3,6,6-tetramethylcyclohexanedione, phenanthraquinone, 1-phenyl-1,2-propanedione and other 1-aryl-2-alkyl-1,2-ethanediones, and cyclic alpha diketones. Most preferred is camphorquinone. Preferred electron donor compounds include substituted amines, e.g., ethyl dimethylaminobenzoate. Other suitable tertiary photoinitiator systems useful for photopolymerizing cationically polymerizable resins are described, for example, in U.S. Pat. No. 6,765,036 (Dede et al.).

Other suitable photoinitiators for polymerizing free radically photopolymerizable compositions include the class of phosphine oxides that typically have a functional wavelength range of 380 nm to 1200 nm. Preferred phosphine oxide free radical initiators with a functional wavelength range of 380 nm to 450 nm are acyl and bisacyl phosphine oxides such as those described in U.S. Pat. No. 4,298,738 (Lechtken et al.), U.S. Pat. No. 4,324,744 (Lechtken et al.), U.S. Pat. No. 4,385,109 (Lechtken et al.), U.S. Pat. No. 4,710,523 (Lechtken et al.), and U.S. Pat. No. 4,737,593 (Ellrich et al.), U.S. Pat. No. 6,251,963 (Kohler et al.); and EP Application No. 0 173 567 A2 (Ying).

Commercially available phosphine oxide photoinitiators capable of free-radical initiation when irradiated at wavelength ranges of greater than 380 nm to 450 nm include bis(2,4,6-trimethylbenzoyl)phenyl phosphine oxide (IRGACURE 819, Ciba Specialty Chemicals, Tarrytown, N.Y.), bis(2,6-dimethoxybenzoyl)-(2,4,4-trimethylpentyl) phosphine oxide (CGI 403, Ciba Specialty Chemicals), a 25:75 mixture, by weight, of bis(2,6-dimethoxybenzoyl)-2,4,4-trimethylpentyl phosphine oxide and 2-hydroxy-2-methyl-1 -phenylpropan-1-one (IRGACURE 1700, Ciba Specialty Chemicals), a 1:1 mixture, by weight, of bis(2,4,6-trimethylbenzoyl)phenyl phosphine oxide and 2-hydroxy-2-methyl-1-phenylpropane-1-one (DAROCUR 4265, Ciba Specialty Chemicals), and ethyl 2,4,6-trimethylbenzylphenyl phosphinate (LUCIRIN LR8893X, BASF Corp., Charlotte, N.C.).

Typically, the phosphine oxide initiator is present in the photopolymerizable composition in catalytically effective amounts, such as from 0.1 weight percent to 5.0 weight percent, based on the total weight of the composition.

Tertiary amine reducing agents may be used in combination with an acylphosphine oxide. Illustrative tertiary amines useful in the invention include ethyl 4-(N,N-dimethylamino)benzoate and N,N-dimethylaminoethyl methacrylate. When present, the amine reducing agent is present in the photopolymerizable composition in an amount from 0.1 weight percent to 5.0 weight percent, based on the total weight of the composition. Useful amounts of other initiators are well known to those of skill in the art.

Suitable photoinitiators for polymerizing cationically photopolymerizable compositions include binary and tertiary systems. Typical tertiary photoinitiators include an iodonium salt, a photosensitizer, and an electron donor compound as described in EP 0 897 710 (Weinmann et al.); in U.S. Pat. No. 5,856,373 (Kaisaki et al.), U.S. Pat. No. 6,084,004 (Weinmann et al.), U.S. Pat. No. 6,187,833 (Oxman et al.), and U.S. Pat. No. 6,187,836 (Oxman et al.); and in U.S. Pat. No. 6,765,036 (Dede et al.). The compositions of the invention can include one or more anthracene-based compounds as electron donors. In some embodiments, the compositions comprise multiple substituted anthracene compounds or a combination of a substituted anthracene compound with unsubstituted anthracene. The combination of these mixed-anthracene electron donors as part of a photoinitiator system provides significantly enhanced cure depth and cure speed and temperature insensitivity when compared to comparable single-donor photoinitiator systems in the same matrix. Such compositions with anthracene-based electron donors are described in U.S. Ser. No. 10/719,598 (Oxman et al.; filed Nov. 21, 2003).

Suitable iodonium salts include tolylcumyliodonium tetrakis(pentafluorophenyl)borate, tolylcumyliodonium tetrakis(3,5-bis(trifluoromethyl)-phenyl)borate, and the diaryl iodonium salts, e.g., diphenyliodonium chloride, diphenyliodonium hexafluorophosphate, diphenyliodonium hexafluoroantimonate, and diphenyliodonium tetrafluoroboarate. Suitable photosensitizers are monoketones and diketones that absorb some light within a range of 450 nm to 520 nm (preferably, 450 nm to 500 nm). More suitable compounds are alpha diketones that have some light absorption within a range of 450 nm to 520 nm (even more preferably, 450 nm to 500 run). Preferred compounds are camphorquinone, benzil, furil, 3,3,6,6-tetramethylcyclohexanedione, phenanthraquinone and other cyclic alpha diketones. Most preferred is camphorquinone. Suitable electron donor compounds include substituted amines, e.g., ethyl 4-(dimethylamino)benzoate and 2-butoxyethyl 4-(dimethylamino)benzoate; and polycondensed aromatic compounds (e.g. anthracene).

The initiator system is present in an amount sufficient to provide the desired rate of hardening (e.g., polymerizing and/or crosslinking). For a photoinitiator, this amount will be dependent in part on the light source, the thickness of the layer to be exposed to radiant energy, and the extinction coefficient of the photoinitiator. Preferably, the initiator system is present in a total amount of at least 0.01 wt-%, more preferably, at least 0.03 wt-%, and most preferably, at least 0.05 wt-%, based on the weight of the composition. Preferably, the initiator system is present in a total amount of no more than 10 wt-%, more preferably, no more than 5 wt-%, and most preferably, no more than 2.5 wt-%, based on the weight of the composition.

Redoxinitiator Systems

In certain embodiments, the compositions of the present invention are chemically hardenable, i.e., the compositions contain a chemically hardenable component and a chemical initiator (i.e., initiator system) that can polymerize, cure, or otherwise harden the composition without dependence on irradiation with actinic radiation. Such chemically hardenable compositions are sometimes referred to as “self-cure” compositions and may include glass ionomer cements, resin-modified glass ionomer cements, redox cure systems, and combinations thereof.

The chemically hardenable compositions may include redox cure systems that include a hardenable component (e.g., an ethylenically unsaturated polymerizable component) and redox agents that include an oxidizing agent and a reducing agent. Suitable hardenable components, redox agents, optional acid-functional components, and optional fillers that are useful in the present invention are described in U.S. Pat. Publication No. 2003/0166740 (Mitra et al.) and 2003/0195273 (Mitra et al.).

The reducing and oxidizing agents should react with or otherwise cooperate with one another to produce free-radicals capable of initiating polymerization of the resin system (e.g., the ethylenically unsaturated component). This type of cure is a dark reaction, that is, it is not dependent on the presence of light and can proceed in the absence of light. The reducing and oxidizing agents are preferably sufficiently shelf-stable and free of undesirable colorization to permit their storage and use under typical dental conditions. They should be sufficiently miscible with the resin system (and preferably water-soluble) to permit ready dissolution in (and discourage separation from) the other components of the hardenable composition.

Useful reducing agents include ascorbic acid, ascorbic acid derivatives, and metal complexed ascorbic acid compounds as described in U.S. Pat. No. 5,501,727 (Wang et al.); amines, especially tertiary amines, such as 4-tert-butyl dimethylaniline; aromatic sulfinic salts, such as p-toluenesulfinic salts and benzenesulfinic salts; thioureas, such as 1-ethyl-2-thiourea, tetraethyl thiourea, tetramethyl thiourea, 1,1-dibutyl thiourea, and 1,3-dibutyl thiourea; and mixtures thereof. Other secondary reducing agents may include cobalt (II) chloride, ferrous chloride, ferrous sulfate, hydrazine, hydroxylamine (depending on the choice of oxidizing agent), salts of a dithionite or sulfite anion, and mixtures thereof. Preferably, the reducing agent is an amine.

Suitable oxidizing agents will also be familiar to those skilled in the art, and include but are not limited to persulfuric acid and salts thereof, such as sodium, potassium, ammonium, cesium, and alkyl ammonium salts. Additional oxidizing agents include peroxides such as benzoyl peroxides, hydroperoxides such as cumyl hydroperoxide, t-butyl hydroperoxide, and amyl hydroperoxide, as well as salts of transition metals such as cobalt (III) chloride and ferric chloride, cerium (IV) sulfate, perboric acid and salts thereof, permanganic acid and salts thereof, perphosphoric acid and salts thereof, and mixtures thereof.

It may be desirable to use more than one oxidizing agent or more than one reducing agent. Small quantities of transition metal compounds may also be added to accelerate the rate of redox cure. In some embodiments it may be preferred to include a secondary ionic salt to enhance the stability of the polymerizable composition as described in U.S. Pat. Publication No. 2003/0195273 (Mitra et al.).

The reducing and oxidizing agents are present in amounts sufficient to permit an adequate free-radical reaction rate. This can be evaluated by combining all of the ingredients of the hardenable composition except for the optional filler, and observing whether or not a hardened mass is obtained.

Preferably, the reducing agent is present in an amount of at least 0.01% by weight, and more preferably at least 0.1% by weight, based on the total weight (including water) of the components of the hardenable composition. Preferably, the reducing agent is present in an amount of no greater than 10% by weight, and more preferably no greater than 5% by weight, based on the total weight (including water) of the components of the hardenable composition.

Preferably, the oxidizing agent is present in an amount of at least 0.01% by weight, and more preferably at least 0.10% by weight, based on the total weight (including water) of the components of the hardenable composition. Preferably, the oxidizing agent is present in an amount of no greater than 10% by weight, and more preferably no greater than 5% by weight, based on the total weight (including water) of the components of the hardenable composition.

The reducing or oxidizing agents can be microencapsulated as described in U.S. Pat. No. 5,154,762 (Mitra et al.). This will generally enhance shelf stability of the hardenable composition, and if necessary permit packaging the reducing and oxidizing agents together. For example, through appropriate selection of an encapsulant, the oxidizing and reducing agents can be combined with an acid-functional component and optional filler and kept in a storage-stable state. Likewise, through appropriate selection of a water-insoluble encapsulant, the reducing and oxidizing agents can be combined with an FAS glass and water and maintained in a storage-stable state.

A redox cure system can be combined with other cure systems, e.g., with a hardenable composition such as described U.S. Pat. No. 5,154,762 (Mitra et al.).

Fillers

The compositions of the present invention can also contain fillers. Fillers may be selected from one or more of a wide variety of materials suitable for incorporation in compositions used for dental applications, such as fillers currently used in dental restorative compositions, and the like.

The filler is preferably finely divided. The filler can have a unimodial or polymodial (e.g., bimodal) particle size distribution. Preferably, the maximum particle size (the largest dimension of a particle, typically, the diameter) of the filler is less than 20 micrometers, more preferably less than 10 micrometers, and most preferably less than 5 micrometers. Preferably, the average particle size of the filler is less than 0.1 micrometers, and more preferably less than 0.075 micrometer.

The filler can be an inorganic material. It can also be a crosslinked organic material that is insoluble in the resin system (i.e., the hardenable components), and is optionally filled with inorganic filler. The filler should in any event be nontoxic and suitable for use in the mouth. The filler can be radiopaque or radiolucent. The filler typically is substantially insoluble in water.

Examples of suitable inorganic fillers are naturally occurring or synthetic materials including, but not limited to: quartz (i.e., silica, SiO₂); nitrides (e.g., silicon nitride); glasses and fillers derived from, for example, Zr, Sr, Ce, Sb, Sn, Ba, Zn, and Al; feldspar; borosilicate glass; kaolin; talc; zirconia; titania; low Mohs hardness fillers such as those described in U.S. Pat. No. 4,695,251 (Randklev); and submicron silica particles (e.g., pyrogenic silicas such as those available under the trade designations AEROSIL, including “OX 50,” “130,” “150” and “200” silicas from Degussa Corp., Akron, OH and CAB-O-SIL M5 silica from Cabot Corp., Tuscola, Ill.). Examples of suitable organic filler particles include filled or unfilled pulverized polycarbonates, polyepoxides, and the like.

Preferred non-acid-reactive filler particles are quartz (i.e., silica), submicron silica, zirconia, submicron zirconia, and non-vitreous microparticles of the type described in U.S. Pat. No.4,503,169 (Randklev). Mixtures of these non-acid-reactive fillers are also contemplated, as well as combination fillers made from organic and inorganic materials.

The filler can also be an acid-reactive filler. Suitable acid-reactive fillers include metal oxides, glasses, and metal salts. Typical metal oxides include barium oxide, calcium oxide, magnesium oxide, and zinc oxide. Typical glasses include borate glasses, phosphate glasses, and fluoroaluminosilicate (“FAS”) glasses. FAS glasses are particularly preferred. The FAS glass typically contains sufficient elutable cations so that a hardened dental composition will form when the glass is mixed with the components of the hardenable composition. The glass also typically contains sufficient elutable fluoride ions so that the hardened composition will have cariostatic properties. The glass can be made from a melt containing fluoride, alumina, and other glass-forming ingredients using techniques familiar to those skilled in the FAS glassmaking art. The FAS glass typically is in the form of particles that are sufficiently finely divided so that they can conveniently be mixed with the other cement components and will perform well when the resulting mixture is used in the mouth.

Generally, the average particle size (typically, diameter) for the FAS glass is no greater than 12 micrometers, typically no greater than 10 micrometers, and more typically no greater than 5 micrometers as measured using, for example, a sedimentation analyzer. Suitable FAS glasses will be familiar to those skilled in the art, and are available from a wide variety of commercial sources, and many are found in currently available glass ionomer cements such as those commercially available under the trade designations VITREMER, VITREBOND, RELY X LUTING CEMENT, RELY X LUTING PLUS CEMENT, PHOTAC-FIL QUICK, KETAC-MOLAR, and KETAC-FIL PLUS (3M ESPE Dental Products, St. Paul, Minn.), FUJI II LC and FUJI IX (G-C Dental Industrial Corp., Tokyo, Japan) and CHEMFIL Superior (Dentsply International, York, Pa.). Mixtures of fillers can be used if desired.

The surface of the filler particles can also be treated with a coupling agent in order to enhance the bond between the filler and the resin. The use of suitable coupling agents include gamma-methacryloxypropyltrimethoxysilane, gamma-mercaptopropyltriethoxysilane, gamma-aminopropyltrimethoxysilane, and the like. Silane-treated zirconia-silica (ZrO₂—SiO₂) filler, silane-treated silica filler, silane-treated zirconia filler, and combinations thereof are especially preferred in certain embodiments.

Other suitable fillers are disclosed in U.S. Pat. No. 6,387,981 (Zhang et al.) and U.S. Pat. No. 6,572,693 (Wu et al.) as well as International Publication Nos. WO 01/30305 (Zhang et al.), WO 01/30306 (Windisch et al.), WO 01/30307 (Zhang et al.), and WO 03/063804 (Wu et al.). Filler components described in these references include nanosized silica particles, nanosized metal oxide particles, and combinations thereof. Nanofillers are also described in U.S. patent application Ser. No. 10/847,781 (Kangas et al.); U.S. patent application Ser. No. 10/847,782 (Kolb et al.); U.S. patent application Ser. No. 10/847,803 (Craig et al.); and U.S. patent application Ser. No. 10/847,805 (Budd et al.) all four of which were filed on May 17, 2004. These applications, in summary, describe the following nanofiller containing compositions:

U.S. patent application Ser. No. 10/847,781 (Kangas et al.) describes stable ionomer compositions (e.g., glass ionomer) containing nanofillers that provide the compositions with improved properties over previous ionomer compositions. In one embodiment, the composition is a hardenable dental composition comprising a polyacid (e.g., a polymer having a plurality of acidic repeating groups); an acid-reactive filler; at least 10 percent by weight nanofiller or a combination of nanofillers each having an average particle size no more than 200 nanometers; water; and optionally a polymerizable component (e.g., an ethylenically unsaturated compound, optionally with acid functionality).

U.S. patent application Ser. No. 10/847,782 (Kolb et al.) describes stable ionomer (e.g., glass ionomer) compositions containing nanozirconia fillers that provide the compositions with improved properties, such as ionomer systems that are optically translucent and radiopaque. The nanozirconia is surface modified with silanes to aid in the incorporation of the nanozirconia into ionomer compositions, which generally contain a polyacid that might otherwise interact with the nanozirconia causing coagulation or aggregation resulting in undesired visual opacity. In one aspect, the composition can be a hardenable dental composition including a polyacid; an acid-reactive filler; a nanozirconia filler having a plurality of silane-containing molecules attached onto the outer surface of the zirconia particles; water; and optionally a polymerizable component (e.g., an ethylenically unsaturated compound, optionally with acid functionality).

U.S. patent application Ser. No. 10/847,803 (Craig et al.) describes stable ionomer compositions (e.g., glass ionomers) containing nanofillers that provide the compositions with enhanced optical translucency. In one embodiment, the composition is a hardenable dental composition including a polyacid (e.g., a polymer having a plurality of acidic repeating groups); an acid-reactive filler; a nanofiller; an optional polymerizable component (e.g., an ethylenically unsaturated compound, optionally with acid functionality); and water. The refractive index of the combined mixture (measured in the hardened state or the unhardened state) of the polyacid, nanofiller, water and optional polymerizable component is generally within 4 percent of the refractive index of the acid-reactive filler, typically within 3 percent thereof, more typically within 1 percent thereof, and even more typically within 0.5 percent thereof.

U.S. patent application Ser. No. 10/847,805 (Budd et al.) describes dental compositions that can include an acid-reactive nanofiller (i.e., a nanostructured filler) and a hardenable resin (e.g., a polymerizable ethylenically unsaturated compound. The acid-reactive nanofiller can include an oxyfluoride material that is acid-reactive, non-fused, and includes a trivalent metal (e.g., alumina), oxygen, fluorine, an alkaline earth metal, and optionally silicon and/or a heavy metal.

For some embodiments of the present invention that include filler (e.g., dental adhesive compositions), the compositions preferably include at least 1% by weight, more preferably at least 2% by weight, and most preferably at least 5% by weight filler, based on the total weight of the composition. For such embodiments, compositions of the present invention preferably include at most 40% by weight, more preferably at most 20% by weight, and most preferably at most 15% by weight filler, based on the total weight of the composition.

For other embodiments (e.g., where the composition is a dental restorative or an orthodontic adhesive), compositions of the present invention preferably include at least 40% by weight, more preferably at least 45% by weight, and most preferably at least 50% by weight filler, based on the total weight of the composition. For such embodiments, compositions of the present invention preferably include at most 90% by weight, more preferably at most 80% by weight, even more preferably at most 70% by weight filler, and most preferably at most 50% by weight filler, based on the total weight of the composition.

Optional Additives

Optionally, compositions of the present invention may contain solvents (e.g., alcohols (e.g., propanol, ethanol), ketones (e.g., acetone, methyl ethyl ketone), esters (e.g., ethyl acetate), other nonaqueous solvents (e.g., dimethylformamide, dimethylacetamide, dimethylsulfoxide, 1-methyl-2-pyrrolidinone)), and water.

If desired, the compositions of the invention can contain additives such as indicators, dyes, pigments, inhibitors, accelerators, viscosity modifiers, wetting agents, buffering agents, stabilizers, and other similar ingredients that will be apparent to those skilled in the art. Viscosity modifiers include the thermally responsive viscosity modifiers (such as PLURONIC F-127 and F-108 available from BASF Wyandotte Corporation, Parsippany, N.J.) and may optionally include a polymerizable moiety on the modifier or a polymerizable component different than the modifier. Such thermally responsive viscosity modifiers are described in U.S. Pat. No. 6,669,927 (Trom et al.) and U.S. Pat. Publication No. 2004/0151691 (Oxman et al.).

Additionally, medicaments or other therapeutic substances can be optionally added to the dental compositions. Examples include, but are not limited to, fluoride sources, whitening agents, anticaries agents (e.g., xylitol), calcium sources, phosphorus sources, remineralizing agents (e.g., calcium phosphate compounds), enzymes, breath fresheners, anesthetics, clotting agents, acid neutralizers, chemotherapeutic agents, immune response modifiers, thixotropes, polyols, anti-inflammatory agents, antimicrobial agents (in addition to the antimicrobial lipid component), antifungal agents, agents for treating xerostomia, desensitizers, and the like, of the type often used in dental compositions. Combination of any of the above additives may also be employed. The selection and amount of any one such additive can be selected by one of skill in the art to accomplish the desired result without undue experimentation.

Preparation and Use of the Compositions

The hardenable dental compositions of the present invention can be prepared by combining an effective amount of an antimicrobial lipid component with a hardenable component using conventional mixing techniques. The resulting composition may optionally contain enhancers, surfactants, fillers, water, co-solvents, and other additives as described herein. In use, the compositions may contain a photoinitiator and be hardened by photoinitiation, or may be hardened by chemical polymerization and contain a redox cure system in which the composition contains an oxidizing agent and a reducing agent. Alternatively, the hardenable composition may contain different initiator systems, such that the composition can be both a photpolymerizable and a chemically polymerizable composition.

The hardenable compositions of the invention can be supplied in a variety of forms including one-part systems and multi-part systems, e.g., two-part powder/liquid, paste/liquid, and paste/paste systems. Other forms employing multi-part combinations (i.e., combinations of two or more parts), each of which is in the form of a powder, liquid, gel, or paste are also possible. In a redox multi-part system, one part typically contains the oxidizing agent and another part typically contains the reducing agent. In multi-part systems containing an antimicrobial lipid component, one part typically contains the antimicrobial lipid component and another part contains either the hardenable component or other components of the final composition. The components of the hardenable composition can be included in a kit, where the contents of the composition are packaged to allow for storage of the components until they are needed.

When used as a dental composition, the components of the hardenable compositions can be mixed and clinically applied using conventional techniques. A curing light is generally required for the initiation of photopolymerizable compositions. The compositions can be in the form of composites or restoratives that adhere very well to dentin and/or enamel. Optionally, a primer layer can be used on the tooth tissue on which the hardenable composition is used. The compositions, e.g., containing a FAS glass or other fluoride releasing material, can also provide very good long-term fluoride release. Some embodiments of the invention may provide glass ionomer cements or adhesives that can be cured in bulk without the application of light or other external curing energy, do not require a pre-treatment, have improved physical properties.

The compositions of the invention are particularly well adapted for use in the form of a wide variety of dental materials, which may be filled or unfilled. They can be used in sealants, coatings, or dental adhesives, which are lightly filled composites (up to 40 wt-% filler, based on the total weight of the composition) or unfilled compositions that are cured after being dispensed adjacent to a tooth (i.e., placing a dental material in temporary or permanent bonding or touching contact with a tooth). They can be used in dental and orthodontic cements, orthodontic adhesives, composites, filling materials, impression materials, and restoratives, which are typically filled compositions (preferably containing greater than 40 wt-% filler and up to 90 wt-% filler).

The compositions can also be used in prostheses that are shaped and polymerized for final use (e.g., as a crown, bridge, veneer, inlay, onlay, or the like), before being disposed adjacent to a tooth. Such preformed articles can be ground or otherwise formed into a custom-fitted shape by the dentist or other user. Although the hardened dental material can be any of a wide variety of materials that are prepared from hardenable components, preferably, the hardened dental material is not a surface pre-treatment material (e.g., etchant or primer). Rather, preferably, the hardened dental material is a restorative (e.g., filling or prosthesis), mill blank, or orthodontic device.

The compositions have utility in clinical applications where cure of conventional light-curable cement may be difficult to achieve. Such applications include, but are not limited to, deep restorations, large crown build-ups, endodontic restorations, attachment of orthodontic brackets (including pre-coated brackets, where, for example, a paste portion could be pre-applied to the bracket and a liquid portion could later be brushed onto a tooth), bands, buccal tubes, and other devices, luting of metallic crowns or other light-impermeable prosthetic devices to teeth, and other restorative applications in inaccessible areas of the mouth.

Preferred compositions are used as dental adhesives, orthodontic adhesives, composites, restoratives, dental cements, orthodontic cements, sealants, coatings, impression materials, filling materials, or combinations thereof.

Objects and advantages of this invention are further illustrated by the following examples, but the particular materials and amounts thereof recited in these examples, as well as other conditions and details, should not be construed to unduly limit this invention. Unless otherwise indicated, all parts and percentages are on a weight basis, all water is deionized water, and all molecular weights are weight average molecular weight.

### EXAMPLES

### Test Methods

Shear Bond Strength Test Method

Adhesive shear bond strength to enamel or dentin for a given test sample was evaluated by the following procedure.

Preparation of Teeth. Bovine incisal teeth, free of soft tissue, were embedded in circular acrylic disks. The embedded teeth were stored in water in a refrigerator prior to use. In preparation for adhesive testing, the embedded teeth were ground to expose a flat enamel or dentin surface using 120-grit sandpaper mounted on a lapidary wheel. Further grinding and polishing of the tooth surface was done using 320-grit sandpaper on the lapidary wheel. The teeth were continuously rinsed with water during the grinding process. The polished teeth were stored in deionized water and used for testing within 2 hours after polishing. The teeth were allowed to warm in a 36° C. oven to between room temperature (23° C.) and 36° C. before use.

Teeth Treatment. Water was applied to the surface of the prepared dry tooth with a brush tip for 10 seconds. An adhesive test sample was then applied with a dental applicator brush to the wet tooth surface with vigorous rubbing for 20 seconds followed by aggressive drying with an air stream for 10 seconds. A second coat of adhesive test sample was then applied for 10 seconds and the adhesive coating was light cured for 10 seconds with an XL 3000 dental curing light (3M Company, St. Paul, Minn.). A 2.5-mm thick Teflon mold with a hole approximately 4.7 mm in diameter was clamped to the embedded tooth such that the hole in the mold exposed part of the adhesively prepared tooth surface. A composite material, A2 shade of FILTEK Z250 Universal Restorative (3M Company), was filled into the hole such that the hole was completely filled, but not overfilled, and light cured for 20 seconds to form a “button” that was adhesively attached to the tooth. The adhesive bond strength of the cured test sample was evaluated after about 24 hours.

Adhesive Bond Strength Testing. The adhesive strength of a cured test sample was evaluated by mounting the assembly (described above) in a holder clamped in the jaws of an INSTRON testing machine (Instron 4505, Instron Corp. Canton, Mass.) with the polished tooth surface oriented parallel to the direction of pull. A loop of orthodontic wire (0.44-mm diameter) was placed around the Z250 button adjacent to the polished tooth surface. The ends of the orthodontic wire were clamped in the pulling jaw of the INSTRON apparatus and pulled at a crosshead speed of 2 mm/min, thereby placing the adhesive bond in shear stress. The force in kilograms (kg) at which the bond failed was recorded, and this number was converted to a force per unit area (units of kg/cm² or MPa) using the known surface area of the button. Each reported value of adhesion to enamel or adhesion to dentin represents the average of 4 to 5 replicates.

Bacteria Kill Rate Test Method

The rate and extent of bacteria kill by a test sample was determined according to the following procedure.

Overnight culture of Streptococcus mutans (S. mutans) (ATCC#25175) in BHI broth (10⁶ CFU/ml) was mixed with a test sample in a Control Solution at a specific concentration for a predetermined time (2, 5, and 10 minutes each). The Control Solution consisted of P-65 surfactant (0.45 parts), isopropyl alcohol (IPA; 4.55 parts), and water (82 parts). Immediately after mixing for the predetermined time, 1.0 ml of the mixture was transferred by pipette into a first tube containing 9.0 ml Letheen Broth to neutralize for fatty acid ester and benzoic acid. This was a 10⁻¹ dilution and was thoroughly mixed with a Vortex mixer. A 1.0-ml aliquot of the 10⁻¹ dilution was transferred by pipette into a second tube containing 9.0 ml Letheen broth and vortexed. This was a 10⁻² dilution. A 0.1-ml aliquot from each of the 10⁻¹ and 10⁻² dilutions was plated out in duplicate and spread on sheep blood agar in Petri dishes with a “hockey stick” applicator. This resulted in 10⁻² and 10⁻³ concentrations of the test sample on each respective plate. The test samples were incubated for 96 hours at 37° C. aerobically followed by counting the number of colony forming units (CFU). This information was compared with the initial inoculum count to determine kill rate for S. mutans at a specified concentration of test sample.

S. Mutans Bacteria Adherence Test Method

S. mutans has the tendency to adhere to only hard surfaces, such as teeth, forming a biofilm or plaque. Such colonization can eventually lead to a number of undesirable clinical side effects that include origination of caries, calcified plaque, irritation of gum tissue leading up to periodontal diseases, etc. Therefore, some of the clinical benefits of using antimicrobial agents in dental materials, such as adhesives or composites, are not only to kill harmful bacteria in the oral cavity but also to suppress the formation of biofilm and secondary caries under a restoration. In this context, the effect of sucrose (metabolized sugars) on plaque formation on cured compositions is valuable. The propensity for S. mutans to form biofilm or plaque on cured discs with and without antimicrobial agents was determined as described in the reference S. Imazato, et al., (J. Dent. Res.; 73(8); 1437-1443; August, 1994. Test sample discs (e.g., Examples 1A and 1B, and Comparative Example CE-1) were prepared by directly casting the test samples (without mixing with water) into discs (15-mm diameter×1-mm thick) under sterile conditions followed by curing (XL 3000 dental curing light) for 80 seconds in air followed by curing (VISIO Beta Light Unit, 3M Company) for 3 minutes under vacuum. Cured discs were also prepared from the commercial products CLEARFIL SE BOND and CLEARFIL PROTECT BOND (both from Kurary Company, Kurashiki, Japan). The Kurary products are 2-part bonding agents that were mixed in equal parts and formed into discs and cured as described above.

The cured discs were each submerged in 12-ml of S. mutans culture (10⁶ CFU/ml) prepared in BHI broth with or without 1% sucrose. Optionally the broth contained 1% sucrose and 1% xylitol, or 1% sucrose and 1% lactoferrin. After 20 hours of incubation at 37° C., the disc samples with accumulated S. mutans plaque were carefully separated from the culture medium suspensions. Each of the discs with biofilm was gently rinsed in water to dislodge loosely held plaque, after which each disc was placed in a test tube containing 5 ml 1 N NaOH and sonicated for 10 minutes to collect the attached plaque. The absorbance of the resulting solution suspensions was determined by conventional spectroscopic measurements of absorbance at 550 nm. Similarly, the culture medium samples (from which the adhesive disc was removed) were also sonicated for 10 minutes followed by absorbance measurements to determine the overall bacterial count. For each test sample, five replicates were tested and the results reported as an average Optical Density (OD) that was assumed to reflect the concentration of S. mutans in the biofilm/plaque or in the culture medium solution.

Bacterial growth of the suspensions without any test sample (i.e., Culture Medium with and without 1% sucrose) were inoculated with bacteria at the same initial concentration and served as a positive control. Control samples also included test samples with no added antimicrobial compositions.

Zone of Inhibition Test Method

S. mutans culture in BHI broth (1 ml; 10⁶ CFU/ml) was uniformly spread over a sheep blood agar with a sterile “hockey stick”. A test sample disc (15-mm diameter×1-mm thick; prepared as described above) was placed at the center of the agar plate and allowed to incubate aerobically for 96 hours at 37° C. The average distance (in mm) between the circumference of the test sample and inside edge of the halo where the bacteria growth was suppressed was determined from six different places and designated as the Inhibition Width. For each test sample, five replicates were tested and the results reported as an average Inhibition Width.

Extended Disinfectant Test Method

A hardened test sample disc (15-mm diameter×1-mm thick; prepared as described above) was allowed to incubate for 72 hours at 37° C. in 9 ml of BHI broth containing about 10⁶ to 10⁸ CFU/ml of S. mutans. After incubation, 1.0 ml of the culture was transferred using a sterile pipette into a tube containing 9.0 ml sterile BHI broth. This was a 10⁻¹ dilution and was thoroughly mixed with a Vortex mixer. A 1.0-ml aliquot of the 10⁻¹ dilution was transferred by pipette into a second tube containing 9.0 ml BHI broth and vortexed. This was a 10⁻² dilution. These steps were repeated through 10⁻⁸ dilutions. A 1.0-ml aliquot from each of the 10⁻¹ through 10⁻⁸ dilutions was plated and spread on sheep blood agar with a “hockey stick” applicator. The samples were incubated for 96 hours at 37° C. aerobically. Colony forming units (CFU/ml) were counted and recorded. Results were reported as Bacteria Count Log Reductions.

Abbreviations, Descriptions, and Sources of Materials

| Abbreviation | Description and Source of Material |
|---|---|
| BHT | 2,6-Di-tert-butyl-4-methylphenol (Sigma-Aldrich, |
| | St. Louis, MO) |
| TEGDMA | Triethyleneglycol dimethacrylate (Sigma-Aldrich) |
| BisGMA | 2,2-Bis[4-(2-hydroxy-3-methacryloyloxypro- |
| | poxy)phenyl]propane CAS No. 1565-94-2 |
| MHP | Methacryloyloxyhexyl phosphate (See Preparation |
| | Method described herein) |
| PM-2 | KAYAMER PM-2; Bis(methacryloxyethyl) phosphate |
| | (Nippon Kayaku, Japan) |
| UDMA | Diurethane dimethacrylate (CAS No. 41137-60-4), |
| | commercially available as Rohamere 6661-0 (Rohm |
| | Tech, Inc., Malden, MA) |
| BisEMA-2 | Six-mole ethoxylated bisphenol A dimethacrylate |
| | (Sartomer CD541, Sartomer Co., Exton, PA) |
| ZrO₂ Filler | Surface-treated zirconia filler (nano-sized |
| | primary particles) (See Preparation Method |
| | described herein) |
| STZ | Silane-treated zirconia-silica filler prepared |
| | as described in U.S. Pat. No. 6,624,211 (Karim) |
| RN-50 | NOIGEN RN-50 polymerizable nonionic surfactant |
| | (DAI -Ichi Kogyo Seiyaku Co. Ltd., Japan; William |
| | H. Minkema, MINK Inc., Plymouth, MN) |
| DPIHFP | Diphenyl iodonium hexafluorophosphate (Johnson |
| | Matthey, Alpha Aesar Division, Ward Hill, NJ) |
| CPQ | Camphorquinone (Sigma-Aldrich) |
| EDMAB | Ethyl 4-(N,N-dimethylamino)benzoate (Sigma- |
| | Aldrich) |
| TPS | Triphenylantimony (Elf Atochem North America, |
| | Philadelphia, PA) |
| GML-12 | Glycerol monolaurate (Med-Chem Labs, Inc., |
| | Galena, IL) |
| PGMC-8 | Propylene glycol monocaprylate (Uniqema, New |
| | Castle, DE) |
| BA | Benzoic acid (Mallinckrodt, St. Louis, MO) |
| SA | Salicylic acid (Sigma-Aldrich) |
| DOSS | Dioctyl sodium sulfosuccinate, anionic surfactant |
| | (Cytec Industries, West Paterson, NJ) |
| P-65 | PLURONIC P-65 nonionic surfactant (Sigma-Aldrich) |
| | (Polyethylene oxide capped polypropylene oxide |
| | having a EO/PO mole ratio of 1 and a molecular |
| | weight of approximately 3400; also available from |
| | BASF Wyandotte Corp. Parsippany, NJ.) |
| Sucrose | Sigma Aldrich |
| Xylitol | Sigma Aldrich |
| Lactoferrin | DMV International, Delhi, NY |
| BHI broth | Brain Heart Infusion broth (VWR, Batavia, IL) |
| Letheen broth | VWR, Batavia, IL |

### Starting Materials Preparations

6-Methacryloyloxyhexyl Phosphate (MHP)

6-Hydroxyhexyl Methacrylate Synthesis: 1,6-Hexanediol (1000.00 g, 8.46 mol, Sigma-Aldrich) was placed in a 1-liter 3-neck flask equipped with a mechanical stirrer and a narrow tube blowing dry air into the flask. The solid diol was heated to 90° C., at which temperature all the solid melted. With continuous stirring, p-toluenesulfonic acid crystals (18.95 g, 0.11 mol) followed by BHT (2.42 g, 0.011 mol) and methacrylic acid (728.49.02 g, 8.46 mol). Heating at 90° C. with stirring was continued for 5 hours during which time vacuum was applied using tap water aspirator for 5-10 minutes after each half-hour reaction time. The heat was turned off and the reaction mixture was cooled to room temperature. The viscous liquid obtained was washed with 10% aqueous sodium carbonate twice (2×240 ml), followed by washing with water (2×240 ml), and finally with 100 ml of saturated NaCl aqueous solution. The obtained oil was dried using anhydrous Na₂SO₄ then isolated by vacuum filtration to give 1067 g (67.70 %) of 6-hydroxyhexyl methacrylate, a yellow oil. This desired product was formed along with 15-18% of 1,6-bis(methacryloyloxyhexane). Chemical characterization was by NMR analysis.

6-Methacryloyloxyhexyl Phosphate (MHP) Synthesis: A slurry was formed by mixing P₄O₁₀ (178.66 g, 0.63 mol) and methylene chloride (500 ml) in a 1-liter flask equipped with a mechanical stirrer under N₂ atmosphere. The flask was cooled in an ice bath (0-5° C.) for 15 minutes. With continuous stirring, 6-hydroxyhexyl methacrylate (962.82 g, which contained 3.78 mol of the mono-methacrylate, along with its dimethacrylate by-product as described above) was added to the flask slowly over 2 hours. After complete addition, the mixture was stirred in the ice bath for 1 hour then at room temperature for 2 hours. BHT (500 mg) was added, and then the temperature was raised to reflux (40-41° C.) for 45 minutes. The heat was turned off and the mixture was allowed to cool to room temperature. The solvent was removed under vacuum to afford 1085 g (95.5%) of 6-Methacryloyloxyhexyl Phosphate (MHP) as a yellow oil. Chemical characterization was by NMR analysis.

Surface-Treated Zirconia (ZrO₂) Filler

Zirconia Sol (217.323 g; 23.5% solids; Nalco, Naperville, Ill.) was weighed into a plastic flask and then added slowly with vigorous stirring to a solution of mono-2-(methacryloyloxy)ethyl succinate (28.796 g; Sigma-Aldrich) in 1-methoxy-2-propanol (200.001 g; Sigma-Aldrich) that was contained in a plastic flask. The resulting mixture was then dried at 90° C. to powder form (dryness) in a convection oven and subsequently ground with a mortar and pestle to a fine powder form for easier later redispersion. Average primary particle size of the zirconia filler was approximately 5 nm, with 50-75 nm loose agglomerates.

Adhesive A

Adhesive A was prepared by combining the ingredients in their relative amounts as shown in Table 1.

TABLE 1
Composition of Adhesive A

| Ingredient | (Parts by Weight) |
|---|---|
| TEGDMA | 31.42 |
| BisEMA-2 | 11.00 |
| MHP | 15.00 |
| PM-2 | 15.00 |
| ZrO₂ | 12.50 |
| UDMA | 11.00 |
| RN-50 | 1.60 |
| CPQ | 1.04 |
| DPIHFP | 0.55 |
| TPS | 0.01 |
| EDMAB | 0.88 |
| TOTAL: | 100 |

Antimicrobial Components A-B and AA-UU

Antimicrobial Components A-B were prepared by combining ingredients in their relative amounts as shown in Table 2A. The Components A or B were then added to Adhesive A to form antimicrobial compositions of the present invention.

The ingredients of Antimicrobial Components AA-UU were individually added to Adhesive A (in their relative amounts as shown in Tables 2A-2D) to form antimicrobial compositions of the present invention.

TABLE 2A
Antimicrobial Components A-B

| Ingredient | A (Parts by Wt.) | B (Parts by Wt.) |
|---|---|---|
| GML-12 | 1.5 | 1.5 |
| PGMC-8 | 1.5 | 1.5 |
| BA | 1.5 | |
| DOSS | 1.0 | 1.0 |
| TOTAL: | 5.5 | 4.0 |

TABLE 2B
Antimicrobial Components AA-HH

| Ingredient | | | | | | | | |
|---|---|---|---|---|---|---|---|---|
| (Parts by Weight) | AA | BB | CC | DD | EE | FF | GG | HH |
| PGMC-8 | 0.25 | 3.0 | 0.25 | 3.0 | 3.0 | 3.0 | 0.25 | 0.25 |
| BA | 1.0 | 0.1 | | | | 1.0 | | 0.1 |
| SA | | | 0.1 | 1.0 | 0.1 | | 1.0 | |
| DOSS | 0.5 | | | 0.5 | 0.01 | | | 0.01 |
| P-65 | | 0.5 | 0.5 | | | 0.01 | 0.01 | |
| TOTAL: | 1.75 | 3.6 | 0.85 | 4.5 | 3.11 | 4.01 | 1.26 | 0.36 |

TABLE 2C
Antimicrobial Components II-PP

| Ingredient | | | | | | | | |
|---|---|---|---|---|---|---|---|---|
| (Parts by Weight) | II | JJ | KK | LL | MM | NN | OO | PP |
| GML-12 | 3.0 | 0.25 | 0.25 | 3.0 | 3.0 | 0.25 | 0.25 | 3.0 |
| BA | | 1.0 | 0.1 | 0.1 | | | | 1.0 |
| SA | 0.1 | | | | 1.0 | 0.1 | 1.0 | |
| DOSS | | | | 0.5 | | 0.5 | 0.01 | 0.01 |
| P-65 | 0.01 | 0.5 | 0.01 | | 0.5 | | | |
| TOTAL: | 3.11 | 1.75 | 0.36 | 3.6 | 4.5 | 0.85 | 1.26 | 4.01 |

TABLE 2D
Antimicrobial Components QQ-UU

| Ingredient | | | | | |
|---|---|---|---|---|---|
| (Parts by Weight) | QQ | RR | SS | TT | UU |
| GML-12 | 0.8125 | 0.8125 | 0.8125 | 0.8125 | 0.8125 |
| PGMC-8 | 0.8125 | 0.8125 | 0.8125 | 0.8125 | 0.8125 |
| BA | 0.275 | 0.275 | 0.275 | 0.275 | 0.275 |
| SA | 0.275 | 0.275 | 0.275 | 0.275 | 0.275 |
| DOSS | 0.1275 | 0.1275 | 0.1275 | 0.1275 | 0.1275 |
| P-65 | 0.1275 | 0.1275 | 0.1275 | 0.1275 | 0.1275 |
| TOTAL: | 2.43 | 2.43 | 2.43 | 2.43 | 2.43 |

### Examples 1A-1B and Comparative Examples (CE) 1-4

### Antimicrobial Adhesive Compositions

Antimicrobial Component A (5.5% by weight) was added to Adhesive A to form an adhesive liquid designated as Example 1A.

Antimicrobial Component B (4.0% by weight) was added to Adhesive A to form an adhesive liquid designated as Example 1B.

Evaluation of Uncured Adhesive Compositions:

Example 1A adhesive composition was evaluated for antimicrobial activity according to the Bacteria Kill Rate Test Method described herein. The results are provided in Table 3 and are compared to results with CE-1 (Adhesive A without added antimicrobial component), CE-2 (Control Solution), and with the commercial antimicrobial product CLEARFIL PROTECT BOND (Kurary Company). The latter is a 2-part bonding agent that was mixed in equal amounts just prior to evaluating. Test solution pH values are also shown in Table 3.

The data in Table 3 show that Example 1A (Adhesive A with 3% GML-12/PGMC-8, 1.5% benzoic acid, and 1% DOSS) had bacterial kill rates equal to or greater than the antimicrobial CLEARFIL PROTECT BOND product and kill rates comparable to CE-1 (Adhesive A Comparative Example with no additionally added antimicrobial agents, but a material known to have inherent antimicrobial activity.)

TABLE 3
Kill Rate Evaluation Results with Adhesives

| | Concentration | | | | | |
|---|---|---|---|---|---|---|
| | (Weight %) of | | Initial | Bacteria Count | | |
| | Adhesive in | | Bacteria | Reduction Log | | |
| | Control | | Count | 2 | 5 | 10 |
| Example | Solution | pH | Log | Min | Min | Min |
| CE-2 | 0 | 3 | 6.61 | 1.06 | 1.06 | 1.06 |
| CE-1 | 0.06 | 3.2 | 5.18 | ≦0.93 | ≦−0.37 | 3.02 |
| CE-1 | 0.26 | 2.7 | 5.18 | ≧3.18 | ≧3.18 | ≧3.18 |
| CLEARFIL | 0.06 | 3.5 | 5.66 | ≦0.12 | ≦0.12 | ≦0.12 |
| PROTECT | | | | | | |
| CLEARFIL | 0.26 | 3.1 | 6.13 | ≧4.13 | ≧4.13 | ≧4.13 |
| PROTECT | | | | | | |
| 1A | 0.06 | 3.1 | 4.49 | ≦−0.84 | ≦−0.05 | 2.03 |
| 1A | 0.26 | 2.7 | 6.13 | ≧4.13 | ≧4.13 | ≧4.13 |

Evaluation of Cured Adhesive Compositions:

Example 1A adhesive composition was evaluated for antimicrobial activity according to the S. mutans Bacteria Adherence Test Method described herein. The results are provided in terms of the test sample optical density as a measure of the presence of S. mutans in biofilm/plaque and in the culture medium (Table 4). The culture medium for the Test Method contained either 0% or 1% sucrose. The results are compared to results with CE-1 (Adhesive A without added antimicrobial component), CE-3 (Culture Medium without added composition), and with the commercial products CLEARFIL SE BOND and CLEARFIL PROTECT BOND (both from Kurary Company). The Kurary products are 2-part bonding agents that were mixed in equal amounts just prior to evaluating.

TABLE 4
Presence of S. Mutans in Plaque and in Culture Medium Samples
with and without added sucrose (Optical Density Values)

| | |
|---|---|
| S. Mutans in | S. Mutans in Culture |
| Plaque | Medium |

| | 0% | 1.0% | 0% | 1.0% |
|---|---|---|---|---|
| Example | Sucrose | Sucrose | Sucrose | Sucrose |
| CE-3 | — | — | 1.0 | 0.3 |
| CE-1 | 0.0 | 0.288 | 0.57 | 0.01 |
| CLEARFIL SE | 0.0 | 0.529 | 0.82 | 0.1 |
| CLEARFIL | 0.0 | 0.273 | 0.84 | 0.13 |
| PROTECT | | | | |
| 1A | 0.0 | 0.157 | 0.5 | 0.064 |

It can be seen from the results shown in Table 4 that plaque is less likely to form on hard surfaces in the absence of sucrose, and that higher bacteria levels were found in culture medium without sucrose. These two distinct observations suggest that the presence of sucrose confines a significant portion of bacteria to biofilm/plaque. In the oral cavity, the presence of sucrose in saliva leads to higher accumulation of bacteria in plaque, which could result in proliferation of caries and, with further neglect, development of serious soft tissue and/or periodontal diseases.

The data of Table 4 also show that Example 1A (Adhesive A with 3% GML-12/PGMC-8, 1.5% benzoic acid, and 1% DOSS) has significantly less propensity to accumulate biofilm/plaque when compared to the Kuraray CLEARFIL products.

Examples 1A and 1B adhesive compositions were evaluated for antimicrobial activity according to the S. Mutans Bacteria Adherence Test Method described herein. The results are provided in terms of the test sample optical density as a measure of the presence of S. mutans in biofilm/plaque (Table 5). The culture medium for the Test Method contained either 1% sucrose, 1% sucrose and 1% xylitol, or 1% sucrose and 1% lactoferrin. The results are compared to results with CE-1 (Adhesive A without added antimicrobial component).

TABLE 5
Presence of S. Mutans in Plaque in the Presence
of Sucrose, Sucrose/Xylitol, or Sucrose/Lactoferin
(Optical Density Values)

| | S. Mutans in Plaque | | |
|---|---|---|---|
| | | 1.0% Sucrose + | 1% Sucrose + |
| Example | 1% Sucrose | 1% Xylitol | 1% Lactoferrin |
| CE-1 | 0.26 | 0.22 | 0.18 |
| 1B | 0.15 | 0.21 | 0.19 |
| 1A | 0.08 | 0.13 | 0.12 |

The data in Table 5 show that Example 1A (Adhesive A with 3% GML-12/PGMC-8, 1.5% benzoic acid, and 1% DOSS) has the greatest antimicrobial activity with significantly less propensity to accumulate biofilm/plaque when compared to CE-1 (Adhesive A with no antimicrobial component).

### Examples 2-22

### Antimicrobial Adhesive Compositions

The ingredients of Antimicrobial Components AA-UU were individually added to Adhesive A in the relative concentrations shown in Tables 2B-2D (total weight percent of the added Antimicrobial Components relative to Adhesive A are shown in Table 6) to form adhesive liquids designated as Examples 2-22 as listed in Tables 6. As above, Adhesive A with no additive was designated Comparative Example 1 (CE-1).

Adhesive A containing antimicrobial components (Examples 2-22) were evaluated for antimicrobial activity according to the Zone of Inhibition Test Method described herein and for Shear Bond Strength (SBS) to dentin and enamel according to the Shear Bond Strength Test Method described herein. The results are provided in Table 6 and are compared to results with CE-1.

It can be concluded from Table 6 that Examples 2-22 all had greater antimicrobial activity (as evidenced by greater Inhibition Width values) than CE-1 and that the presence of the Antimicrobial Components AA-UU in Adhesive A does not significantly affect the shear bond strength of Adhesive A on enamel and dentine.

TABLE 6
Antimicrobial Adhesive Compositions.
Results of Antimicrobial and Shear Bond Strength (SBS) Evaluations

| | Adhesive A + | | | | |
|---|---|---|---|---|---|
| | Antimicrobial | | | | |
| | Component (AMC) | | Inhibition | SBS, | SBS, |
| | | % By | Width | Enamel | Dentine |
| Ex. | AMC | Weight AMC | (mm) | (MPa) | (MPa) |
| 2 | AA | 1.720 | 2.06 | 21.38 | 14.12 |
| 3 | BB | 3.475 | 2.46 | 25.04 | 10.81 |
| 4 | CC | 0.843 | 2.65 | 19.21 | 14.75 |
| 5 | DD | 4.306 | 2.73 | 22.75 | 16.29 |
| 6 | EE | 3.016 | 2.84 | 22.3 | 15.4 |
| 7 | FF | 3.855 | 2.93 | 23.3 | 11.3 |
| 8 | GG | 5.063 | 3.41 | 26.5 | 16.4 |
| 9 | HH | 0.359 | 3.79 | 20.4 | 14.9 |
| 10 | II | 3.016 | 2.22 | 22.07 | 10.92 |
| 11 | JJ | 1.720 | 4.47 | 26.30 | 13.09 |
| 12 | KK | 0.359 | 2.38 | 18.0 | 11.5 |
| 13 | LL | 3.475 | 2.9 | 19.7 | 12.7 |
| 14 | MM | 4.306 | 1.15 | 17.7 | 14.5 |
| 15 | NN | 0.843 | 3.1 | 22.4 | 13.1 |
| 16 | OO | 1.244 | 2.94 | 21.5 | 18.6 |
| 17 | PP | 3.855 | 3.1 | 27.2 | 10.8 |
| 18 | QQ | 2.372 | 2.2 | 24.47 | 11.09 |
| 19 | RR | 2.372 | 2.25 | 20.24 | 10.98 |
| 20 | SS | 2.372 | 2.79 | 23.90 | 11.32 |
| 21 | TT | 2.372 | 2.87 | 23.4 | 16.1 |
| 22 | UU | 2.372 | 3.72 | 23.3 | 13.5 |
| CE-1 | None | — | 1.47 | 26.1 | 17.2 |

### Example 23

### Composite Containing Antimicrobial Component

Antimicrobial Component A was added at levels of 0%, 1.75% and 5.0% by weight (based on total weight of the composite) to the Base-side of PROTEMP II Composite for Temporary Crowns and Bridges (3M ESPE) to form Control Example 23A and Examples 23B and 23C, respectively. Control Example 23A and Examples 23A-23B were evaluated for antimicrobial activity according to the Extended Disinfectant Test Method described herein and the results are reported in Table 7.

### Example 24

### Luting Cement Containing Antimicrobial Component

Antimicrobial Component A was added at levels of 0%, 1.0%, 2.5% and 5.0% by weight (based on total weight of the cement) to the Liquid-side of RELY X Luting Cement (3M ESPE) to form Control Example 24A and Examples 24B, 24C, and 24D, respectively. Control Example 24A and Examples 24B-24D were evaluated for antimicrobial activity according to the Extended Disinfectant Test Method described herein and the results are reported in Table 7.

### Example 25

### Universal Restorative Containing Antimicrobial Component

Antimicrobial Component A was added at levels of 0%, 1.0%, 2.5% and 5.0% by weight (based on total weight of the restorative) to the Resin-side of FILTEK SUPREME Universal Restorative (3M ESPE) to form Control Example 25A and Examples 25B, 25C, and 25D, respectively. Control Example 25A and Examples 25B-25D were evaluated for antimicrobial activity according to the Extended Disinfectant Test Method described herein and the results are reported in Table 7.

Control Example 25A and Examples 25B-25D were also evaluated for antimicrobial activity according to the S. mutans Bacteria Adherence Test Method described herein and the results are reported as follows in terms of the test sample optical density as a measure of the presence of S. mutans in biofilm/plaque for different concentrations of Antimicrobial Component A in Adhesive A:

| | Concentration of Antimicrobial Component A | |
|---|---|---|
| Ex. | (Weight %) | Optical Density |
| 25A | 0 | 0.154 |
| 25B | 1.0 | 0.161 |
| 25C | 2.5 | 0.125 |
| 25D | 5.0 | 0.061 |

### Example 26

### GI Liner/Base Containing Antimicrobial Component

Antimicrobial Component A was added at levels of 0%, 1.25%, 2.5% and 5.0% by weight (based on total weight of the liner/base) to the Liquid-side of VITREBOND Light Cure Glass lonomer (GI) Liner/Base (3M ESPE) to form Control Example 26A and Examples 26B, 26C, and 26D, respectively. Control Example 26A and Examples 26B-26D were evaluated for antimicrobial activity according to the Extended Disinfectant Test Method described herein and the results are reported in Table 7.

### Example 27

### Impression Material Containing Antimicrobial Component

Antimicrobial Component A was added at levels of 0%, 1.3%, 2.7% and 4.9% by weight (based on total weight of the impression material) to IMPRINT II Impression Material (3M ESPE) to form Control Example 27A and Examples 27B, 27C, and 27D, respectively. Control Example 27A and Examples 27B-27D were evaluated for antimicrobial activity according to the Extended Disinfectant Test Method described herein and the results are reported in Table 7.

TABLE 7
Antimicrobial Adhesive Compositions.
Results of Antimicrobial Evaluations (Extended Disinfectant Test)

| | | % By Weight | Bacteria Count |
|---|---|---|---|
| Example | Dental Material | Component A | Reduction (Log) |
| 23A | PROTEMP II Composite | 0 | 0.06 |
| 23B | PROTEMP II Composite | 1.7 | 1.26 |
| 23C | PROTEMP II Composite | 5.0 | 8.02 |
| 24A | RELY X Luting Cement | 0 | 1.84 |
| 24B | RELY X Luting Cement | 1.0 | 3.42 |
| 24C | RELY X Luting Cement | 2.5 | 3.55 |
| 24D | RELY X Luting Cement | 5.0 | 8.02 |
| 25A | FILTEK SUPREME | 0 | 6.44 |
| | Restorative | | |
| 25B | FILTEK SUPREME | 1.0 | 6.42 |
| | Restorative | | |
| 25C | FILTEK SUPREME | 2.5 | 6.44 |
| | Restorative | | |
| 25D | FILTEK SUPREME | 5.0 | 5.89 |
| | Restorative | | |
| 26A | VITREBOND GI Liner/ | 0 | 8.02 |
| | Base | | |
| 26B | VITREBOND GI Liner/ | 1.25 | 8.02 |
| | Base | | |
| 26C | VITREBOND GI Liner/ | 2.5 | 8.02 |
| | Base | | |
| 26D | VITREBOND GI Liner/ | 5.0 | 8.02 |
| | Base | | |
| 27A | IMPRINT II | 0 | 1.58 |
| | Impression Material | | |
| 27B | IMPRINT II | 1.3 | 1.07 |
| | Impression Material | | |
| 27C | IMPRINT II | 2.7 | 1.09 |
| | Impression Material | | |
| 27D | IMPRINT II | 4.9 | 1.93 |
| | Impression Material | | |

The results in Table 7 show that the addition of Antimicrobial Component A to various Dental Materials generally imparts a dose-dependent increase in antibacterial activity to the Materials. In the case of FILTEK SUPREME Restorative and VITREBOND GI Liner/Base, these two Dental Materials were found to be inherently antibacterial even without the addition of Antimicrobial Component A.

The complete disclosures of the patents, patent documents, and publications cited herein are incorporated by reference in their entirety as if each were individually incorporated. Various modifications and alterations to this invention will become apparent to those skilled in the art without departing from the scope and spirit of this invention. It should be understood that this invention is not intended to be unduly limited by the illustrative embodiments and examples set forth herein and that such examples and embodiments are presented by way of example only with the scope of the invention intended to be limited only by the claims set forth herein as follows.

What is claimed is:

1. A dental composition comprising:
an effective amount of an antimicrobial lipid component comprising a (C7-C12)saturated fatty acid ester of a polyhydric alcohol, a (C8-C22)unsaturated fatty acid ester of a polyhydric alcohol, a (C7-C12)saturated fatty ether of a polyhydric alcohol, a (C8-C22)unsaturated fatty ether of a polyhydric alcohol, an alkoxylated derivative thereof, or combinations thereof, wherein the alkoxylated derivative has less than 5 moles of alkoxide per mole of polyhydric alcohol; with the proviso that for polyhydric alcohols other than sucrose, the esters comprise monoesters and the ethers comprise monoethers, and for sucrose the esters comprise monoesters, diesters, or combinations thereof, and the ethers comprise monoethers, diethers, or combinations thereof; and
a hardenable component.

2. The dental composition of claim 1 further comprising an effective amount of an enhancer component distinct from the antimicrobial lipid component.

3. The dental composition of claim 2 wherein the enhancer component comprises a carboxylic acid.

4. The dental composition of claim 2 wherein the enhancer component comprises an alpha-hydroxy acid.

5. The dental composition of claim 2 wherein the enhancer component comprises an alpha-hydroxy acid, a beta-hydroxy acid, a chelating agent, a (C1-C4)alkyl carboxylic acid, a (C6-C12)aryl carboxylic acid, a (C6-C12)aralkyl carboxylic acid, a (C6-C12)alkaryl carboxylic acid, a phenolic compound, a (C1-C10)alkyl alcohol, an ether glycol, or combinations thereof.

6. The dental composition of claim 2 wherein the total concentration of the enhancer component relative to the total concentration of lipid component is within a range of 10:1 to 1:300, on a weight basis.

7. The dental composition of claim 1 further comprising an effective amount of a surfactant component distinct from the antimicrobial lipid component.

8. The dental composition of claim 7 wherein the surfactant component comprises a sulfonate surfactant, a sulfate surfactant, a phosphonate surfactant, a phosphate surfactant, a poloxamer surfactant, a cationic surfactant, or mixtures thereof.

9. The dental composition of claim 8 wherein the surfactant component comprises a sulfonate surfactant, a sulfate surfactant, a poloxamer surfactant, or mixtures thereof.

10. The dental composition of claim 9 wherein the surfactant component is dioctyl sodium sulfosuccinate.

11. The dental composition of claim 9 wherein the surfactant component is a poloxamer comprising a copolymer of polyethylene oxide and polypropylene oxide.

12. The dental composition of claim 7 wherein the total concentration of the surfactant component to the total concentration of antimicrobial lipid component is within a range of 5:1 to 1:100, on a weight basis.

13. The dental composition of claim 1 wherein the hardenable component comprises acid functionality.

14. The dental composition of claim 13 wherein the acid functionality comprises carboxylic acid functionality, phosphoric acid functionality, phosphonic acid functionality, sulfonic acid functionality, or combinations thereof.

15. The dental composition of claim 1 further comprising an initiator system.

16. The dental composition of claim 1 wherein the hardenable component comprises an ethylenically unsaturated compound.

17. The dental composition of claim 16 wherein the ethylenically unsaturated compound is selected from the group consisting of an ethylenically unsaturated compound with acid functionality, an ethylenically unsaturated compound without acid functionality, and combinations thereof.

18. The dental composition of claim 16 wherein the ethylenically unsaturated compound is a (meth)acrylate compound.

19. The dental composition of claim 1 wherein the hardenable component comprises a glass ionomer cement.

20. The dental composition of claim 19 wherein the glass ionomer cement is a resin-modified glass ionomer cement.

21. The dental composition of claim 1 wherein the hardenable component comprises a polyether, a polysiloxane, or combinations thereof.

22. The dental composition of claim 1 wherein the hardenable component comprises an epoxide, a vinyl ether, or combinations thereof.

23. The dental composition of claim 1 wherein the antimicrobial lipid component comprises glycerol monolaurate, glycerol monocaprate, glycerol monocaprylate, propylene glycol monolaurate, propylene glycol monocaprate, propylene glycol monocaprylate, or combinations thereof.

24. The dental composition of claim 1 wherein the antimicrobial lipid component is present in an amount of at least 0.1 wt-%.

25. The dental composition of claim 24 wherein the antimicrobial lipid component comprises a monoester of a polyhydric alcohol, a monoether of a polyhydric alcohol, or an alkoxylated derivative thereof, and the antimicrobial lipid component further includes no greater than 15 wt-%, based on the total weight of the antimicrobial lipid component, of a di- or tri-ester, a di- or tri-ether, alkoxylated derivative thereof, or combinations thereof.

26. The dental composition of claim 1 further comprising a filler.

27. The dental composition of claim 1 wherein the composition is selected from the group consisting of dental adhesives, orthodontic adhesives, composites, restoratives, dental cements, orthodontic cements, sealants, coatings, impression materials, filling materials, and combinations thereof.

28. A method of preparing a dental article, the method comprising:
combining an antimicrobial lipid component and a hardenable component to form the dental composition of claim 1; and
hardening the composition to fabricate a dental article selected from the group consisting of crowns, bridges, veneers, inlays, onlays, fillings, mill blanks, impression materials, orthodontic devices, prostheses, and finishing or polishing devices.
